SEQUENCE LISTING (9 sequences)

SEQ ID NO: 1 - DNA, 58 bp, Artificial Sequence
  Description of Artificial Sequence  Synthetic Primer
ggcagctttacgatgtacattcaagagatgtacatcgtaaagctgccttttttccaat

SEQ ID NO: 2 - DNA, 58 bp, Artificial Sequence
  Description of Artificial Sequence  Synthetic Primer
catttgttccaagctccagttcaagagactggagcttggaacaaatgttttttccaat

SEQ ID NO: 3 - DNA, 21 bp, Artificial Sequence
  Description of Artificial Sequence  Synthetic Primer
agccagacttccggagcgatc

SEQ ID NO: 4 - DNA, 22 bp, Artificial Sequence
  Description of Artificial Sequence  Synthetic Primer
acttggcaaagcgggtgaaacg

SEQ ID NO: 5 - DNA, 20 bp, Artificial Sequence
  Description of Artificial Sequence  Synthetic Primer
cctggcaggcaatgcggtcc

SEQ ID NO: 6 - DNA, 19 bp, Artificial Sequence
  Description of Artificial Sequence  Synthetic Primer
gggcaggtggtacgttgtg

SEQ ID NO: 7 - DNA, 24 bp, Artificial Sequence
  Description of Artificial Sequence  Synthetic Primer
cgtaaagctgccttctgttttttt

SEQ ID NO: 8 - DNA, 845 bp, Homo sapiens
caggtgagcctctcactcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaggccaaaaagaaa

SEQ ID NO: 9 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

(19) United States
(12) Patent Application Publication
(10) Pub. No.: US 2008/0274104 A1
(43) Pub. Date: Nov. 6, 2008

(54) Targeting a Secreted Pro-Apoptotic Factor for Cancer Therapeutics

(76) Inventors: Ralph B. Arlinghaus, Bellaire, TX (US); Xie Shanhai, Houston, TX (US); Hui Lin, Houston, TX (US); Sun Tong, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 11/816,011

(22) PCT Filed: Feb. 10, 2006
(86) PCT No.: PCT/US06/04748

§ 371 Date: Apr. 7, 2008

Related U.S. Application Data
(60) Provisional application No. 60/651,877, filed on Feb. 10, 2005.

(51) Int. Cl. A61K 39/395; G01N 33/53; A61K 31/7088; A61P 35/00

(52) U.S. Cl. 424/133.1; 424/145.1; 435/7.1; 514/44

(57) ABSTRACT

The present invention concerns targeting a cell death factor associated with cancer. More specifically, an apoptosis-inducing factor is targeted to prevent destruction of non-cancerous cells. The factor may be a lipocalin molecule, and in specific embodiments its secretion and/or the secreted form is targeted by an inhibitory agent, such as an antibody, small molecule, antisense RNA, or siRNA, for example.

The present invention is filed under 35 U.S.C. §317, claiming priority to PCT/US2006/004748, filed Feb. 10, 2006, and also claims priority to U.S. Provisional Patent Application Ser. No. 60/651,877, filed Feb. 10, 2005, all of which applications are incorporated by reference herein in their entirety.

### STATEMENT REGARDING FEDERALLY SPONSORED RESEARCH OR DEVELOPMENT

The present invention was generated by funds from the National Institutes of Health Grant Nos. CA49639 and CA16672. The United States Government has certain rights in the invention.

### FIELD OF THE INVENTION

The present invention concerns at least the fields of molecular biology, cell biology, cancer biology, and medicine. In particular embodiments, the field of the invention concerns targeting secretion of a cell death factor

### BACKGROUND OF THE INVENTION

Lipocalins are a diverse class of secreted glycoproteins that have been widely studied. 24p3 is also known as SIP24 (Davis et al., 1991), uterocalin (Liu et al., 1997) and neutrophil gelatinase-associated lipocalin (NGAL) (human form) (Kjeldsen et al., 1993; Kjeldsen et al., 2000). Lipocalins function in a variety of processes including nutrient transport (Flower et al., 1991), binding of ferric iron structures (Goetz et al., 2002) and immune homeostasis (Devireddy et al., 2001). Members of the lipocalin family have sequence conservation; sequence similarity between family members (for example mouse and human) involves conserved cysteines and three motifs involved in target cell recognition (Kjeldsen et al., 2000).

Devireddy et al. (2001) detected increased 24p3 gene expression following IL-3 withdrawal in mouse FL5.12 cells. They further showed that mouse 24p3 induces apoptosis in normal blood cells including murine primary bone marrow, splenocytes, thymocytes, and human neutrophils and peripheral blood lymphocytes. Various mouse IL-3 dependent cell lines, such as 32D cells, are also susceptible to 24p3 apoptotic effects. Apoptosis induction by secreted 24p3 involves dephosphorylation of the proapoptotic protein Bad in the targeted cell (Devireddy et al., 2001).

### SUMMARY OF THE INVENTION

The present invention concerns targeting a molecule that is deleterious to a non-cancerous cell. In particular, when a non-cancerous cell is subjected to a particular molecule, this results in the destruction of the non-cancerous cell, such as by apoptosis, for example. In particular aspects of the invention, the destruction of one or more non-cancerous cells thereby permits or facilitates proliferation of one or more cancerous cells. In specific embodiments, the cancerous cells are able to overgrow in a particular tissue by providing space through removal of the non-cancerous cells. In a specific aspect of the invention, the molecule is secreted by the cancerous cell, and in certain embodiments this may also be referred to as being released from the cancerous cell.

In embodiments of the invention, the molecule is targeted in one or more ways, including by targeting its secretion from the cell, by targeting its production, by targeting it following secretion from the cell, including by targeting its activity and/or preventing it from acting on a non-cancerous cell, or a combination thereof, for example. In specific embodiments, the non-cancerous cell-destroying molecule is bound by another molecule such that the non-cancerous cell destroying molecule is no longer capable of acting on the non-cancerous cell. In specific embodiments of the invention, the non-cancerous cell-destroying molecule is lipocalin and the molecule to target it is a small molecule, an antibody, such as a monoclonal antibody, and including a humanized antibody, a polypeptide, a peptide, and so forth. In other aspects of the invention, the expression of the non-cancerous cell-destroying molecule is targeted, such as by antisense RNA, siRNA, or both.

In particular embodiments, the secreted form of lipocalin may be modified, and a skilled artisan recognizes that an exemplary modified form of lipocalin in humans is referred to as NGAL, whereas an exemplary modified form of lipocalin in mice is referred to as 24p3. The term “modified” may refer to the lipocalin (NGAL) being 21 kDa, as opposed to the 24 kDa form of wild-type NGAL. The lipocalin may be a glycoprotein, and in particular embodiments of the invention it is glycosylated, whereas in alternative embodiments it is not glycosylated. In one particular embodiment of the invention, the secreted lipocalin is a modified form such that it results from alternative splicing of the polynucleotide that expresses it, such as, for example, deletion of part or all of one or more exons.

The structure of the secreted lipocalin may be identified by standard means in the art, such as by purifying the protein on a substrate, such as a gel, and then performing mass spectrometry, for example.

In particular aspects of the invention, lipocalin acts alone in affecting a non-cancerous cell, whereas in alternative embodiments lipocalin acts in conjunction with one or more molecules to affect a non-cancerous cell. Agents used in the invention to target lipocalin may also target other molecules that act in conjunction with lipocalin. In other embodiments, agents used in the invention may target only molecules that act in conjunction with lipocalin and do not directly target lipocalin, for example.

In specific embodiments, the cancerous cell is a leukemia cell, such as a chronic myeloid leukemia cell, for example. The cell may be further defined as a BCR-ABL cell, wherein there is the presence of the BCR-ABL oncogene product (a tyrosine kinase), particularly in a cell that induces atrophy or reduces hematopoiesis. Suppression of normal hematopoiesis permits the leukemia cell from resisting immune responses that can destroy the leukemic cell. In particular embodiments, the secreted lipocalin results in the indirect or direct death of normal bone marrow and spleen cells, which permits leukemic cells to invade the marrow and spleen. In other embodiments, the cancer may be a primary cancer originating in another tissue, and those cells that secrete lipocalin are suitable for invading bone marrow, for example. Thus, the primary cancers of the individual may be breast, prostate, liver, spleen, pancreatic, stomach, kidney, ovarian, cervical, brain, leukemia, melanoma, gall bladder, head and neck, esophageal and so forth. For example, if it is to be determined whether or not lipocalin-secreting cells are present in bone marrow, bone marrow aspirate may be obtained and determined if lipocalin is present therein. In other words, one can determine if lipocalin is secreted from cancer cells in the bone marrow, and a correlation may be made to there being metastatic cancer cells in the bone marrow from a primary tissue that is not bone marrow.

One of skill in the art may assay for a lipocalin-secreting cell by assaying the medium of a cell suspected thereof for the presence of lipocalin. A skilled artisan recognizes that normal cells secrete lipocalin upon certain physiological signals known in the art, such as starvation, bacterial infection, and so forth, and in particular embodiments the lipocalin of the present invention is a modified form that is detectable upon standard assays in the art. Such an assay may reflect the mass or size of the lipocalin, such as by running the lipocalin on a gel or column, for example.

Given that normal cells produce lipocalin but do not persistently secrete it, in particular embodiments the present invention concerns secreted lipocalin and cells that secrete lipocalin (including a modified form of lipocalin) as being determinate of a cancer cell and providing a target to treat the cancer, more particularly from protecting normal cells from premature death by lipocalin. In specific embodiments, the secreted lipocalin does not adversely affect the cancerous cells. Thus, in particular embodiments the present invention may or may not prevent proliferation of cancerous cells, and an individual may be provided additional therapy to target the cancerous cells themselves. The cancerous cells may be targeted by any suitable means, such as by chemotherapy, transplant, surgery, radiation, hormone therapy, gene therapy, immunotherapy, a combination thereof, and so forth. Particular additional chemotherapeutics, such as those suitable for leukemia, include Gleevec, interferon, busulfan, a combination thereof, and so forth.

In specific aspects of the invention, chronic myeloid leukemia is at least a two step process: 1) oncogenic transformation of blood cells that causes leukemia; and 2) destruction of normal blood cells by a factor secreted by leukemia cells to which they themselves are resistant, allowing a few newly formed leukemic cells to establish themselves in the highly active normal marrow environment. In further specific embodiments, the cell death factors secreted by leukemia cells depress the immune responses that would destroy the newly formed leukemia cells produced as a result of the Philadelphia chromosome formation (which causes production of the Bcr-Abl oncoprotein). Thus, in particular aspects of the invention, the lipocalin-secreting cancer cells, such as leukemic cells, are not just outgrowing the neighboring non-cancerous cells but take a positive action by the activity of lipocalin to negatively affect the non-cancerous cells and facilitate or permit their cancerous proliferation. Embodiments of the present invention include affecting the outgrowing of the cancer cells compared to the neighboring non-cancerous cells and/or affecting the negative effect of lipocalin on the neighboring non-cancerous cells.

In specific aspects of the invention, the methods and compositions are useful for individuals with leukemia, including chronic myeloid leukemia. It is well-known that human leukemia is characterized by the proliferation of abnormal white blood cells that lead to a cancer-like state in which leukemia cells accumulate in abnormal sites such as lung and spinal cord. A second change in the bone marrow comprises atrophic changes, which are diagnosed as atrophy, hypoplasia and depletion of normal blood cells. The present inventors discovered that leukemia cells expressing the BCR-ABL oncogene secrete a cell death factor, 24p3. 24p3 is a lipocalin secreted by normal mouse hematopoetic cells deprived of the cytokine IL-3. This factor induces cell death in populations of 32D cells maintained in medium with or without IL-3. The present inventors found that Bcr-Abl expressing 32D cells express 24p3 RNA as do 32D cells deprived of IL-3, but as expected 32D cells maintained in IL-3 lack 24p3 expression. Importantly, 32D cells expressing the Bcr-Abl oncoprotein, although producing 24p3, are resistant to its cell death effects. Conditioned medium from COS cells expressing 24p3 caused increased apoptosis in normal mouse bone marrow cells maintained in primary culture as well as IL-3 dependent 32D cells maintained in IL-3 or not, but Bcr-Abl positive 32D cells were resistant to these apoptotic effects. Moreover, the results indicate that level of IL-3 in the tissue environment is a resistant factor that counteracts 24p3. This situation tends to select for leukemic cells, especially those with high levels of the Bcr-Abl oncoprotein.

These findings indicate, in specific embodiments of the invention, that the long-observed depression of nominal hematopoieisis in leukemia patients is an active process involving lipocalins or other cell death-inducing factors produced by the leukemic cell clones. Thus, leukemic clones, although producing a cell death factor, are resistant to its apoptotic effects in contrast to normal hematopoietic cells that would undergo apoptosis upon exposure to the factor.

Injection of human leukemia cells into NOD (scid) mice suppresses mouse hematopoiesis. Injection of the K6 clone of K562 cells containing a silenced form of a BCR cDNA gene (a Tet-off BCR cDNA) causes a wasting syndrome and death in 100% of the mice within 35 days. Pathology studies revealed that these mice have two syndromes. One involves tumors of the spleen and marrow. However, these neoplastic changes are not associated with increases in the levels of circulating white blood cells. The second syndrome was very interesting, as most mice had a depletion of mouse hematopoietic cells within their spleens and marrow. The present inventors investigated the cause of this depression in normal mouse hematopoiesis. Importantly, the present inventors did not observe these effects in mice in which expression of the BCR gene was stimulated by removal of the Tet block, nor was depression of mouse hematopoiesis observed in mice that received only the conditioning dose of radiation to facilitate grafting of the human cells (no injection of human leukemia cells). In contrast, the spleens and marrow of these mice had a vigorous amount of normal hematopoiesis. In a specific embodiment of the invention, the injected K562 K6 cell clone secretes some factor into the tissue environment that appears to induce a repressive factor that severely reduces the level of normal diploid mouse hematopoietic cells. In further specific embodiments, the repressive factor comprises lipocalin.

In specific embodiments of the invention, the effect of antisense 24p3 on preventing atrophy of normal bone marrow is observed. First, both antisense 24p3 and sense 24p3 plasmid are introduced into Bcr-Abl expressing 32D cells. Then, Bcr-Abl positive 32D cells, Bcr-Abl positive 32D cells expressing antisense 24p3, Bcr-Abl positive 32D cells expressing sense 24P3, and 32D cells are injected into NOD/Scid mice. After mice are dead or sacrificed, the bone marrow, liver and spleen are collected for pathological analysis. Improvement of atrophy phenomenon indicates the antisense 24p3 is useful in gene therapy.

In particular embodiments of the invention, there is treatment of CML. In specific embodiments, the treatment comprises use of a humanized anti-NGAL antibody, and in further specific embodiments there is injection of the antibody in early stage CML patients to reverse/retard invasion of marrow and spleen and to enhance immune responses directed towards leukemia cells. In certain aspects, the antibody is injected, such as intravenously or intramuscularly, for example. Individuals receive forms of therapy that inhibit the Bcr-Abl oncoprotein (e.g., Gleevec), in particular embodiments. Frequency and dosage is determined by mouse and human studies, as is routine in the art.

In one embodiment of the invention, there is a method of inhibiting secretion of lipocalin from a cell and/or targeting a secreted lipocalin from a cell of an individual, comprising the step of administering to the individual a therapeutically effective amount of a lipocalin-inhibiting substance. The lipocalin-inhibiting substance may comprise a small molecule, an antibody, a DNA, an RNA, a polypeptide, a peptide, a combination thereof, or a mixture thereof. The lipocalin may be a modified form of lipocalin, such as one that is about 21 kDa, for example. In particular embodiments of the invention, the administering step comprises injection.

Lipocalin-inhibiting substances that comprise an antibody include a monoclonal antibody, a humanized antibody, or both, for example. Lipocalin-inhibiting substances may comprise antisense RNA, siRNA, or both. Lipocalin-inhibiting substances may be identified by exemplary screening methods of the invention, including those provided herein. In a specific embodiment, the individual has cancer, such as leukemia (for example chronic myeloid leukemia), breast cancer, or prostate cancer, for example.

The lipocalin-inhibiting substance may comprise a vector, including a viral vector or a non-viral vector (such as a plasmid). Exemplary viral vectors comprise a lentiviral vector, a retroviral vector, an adenoviral vector, or an adeno-associated vector.

In an embodiment of the invention, there is a method of screening for an agent that inhibits secretion of lipocalin from a cell or that targets secreted lipocalin from a cell. Any suitable screen may be employed, although in specific embodiments the method comprises the step of determining whether or not a test compound modulates the secretion of lipocalin, such as secretion of a modified form of lipocalin, and/or whether or not a test compound modulates a secreted lipocalin, such as a modified form of a secreted lipocalin. Specific steps may include providing a cell that secretes lipocalin into a medium; providing a test compound to the cell, the medium or both; and assaying the medium for the presence of lipocalin, assaying modulation of lipocalin by the test compound, or both, for example. In a specific embodiment the test compound is an antibody, a small molecule, a nucleic acid, a polypeptide, a peptide, or a mixture thereof, for example. The antibody may be a monoclonal antibody, for example. In a specific embodiment, the nucleic acid is antisense RNA, siRNA, or both, for example.

Cells to be employed in screens may be any cells that secrete lipocalin, although in particular embodiments they are cells that secrete a modified form of lipocalin, such as, for example, one that is about 21 kDa. In specific embodiments, the cell is a leukemia cell, such as, for example, a chronic myeloid leukemia cell.

In particular embodiments, a therapeutically effective amount of an agent identified by a screen of the present invention is delivered to an individual with cancer. In specific embodiments, the cancer is leukemia, such as chronic myeloid leukemia, for example. In further specific embodiments, the individual is treated with an additional cancer therapy, such as, for example, chemotherapy, radiation, surgery, gene therapy, hormone therapy, immunotherapy, or a combination thereof.

In additional embodiments of the invention, there is an agent that prevents interaction (whether direct or indirect) of lipocalin with a non-cancerous cell, such as a hematopoietic cell. The agent may be referred to as an anti-lipocalin agent, a lipocalin-inhibiting agent, a lipocalin-targeting agent, or an anti-secreted lipocalin agent, for example. In specific embodiments, the agent is comprised in a pharmaceutically acceptable excipient, a liposome, or both, for example.

In another embodiment of the invention, there is a method of protecting a non-cancerous cell from destruction by a cancer cell, comprising the step of delivering to the cancer cell an agent identified by a screening method of the present invention. In another embodiment of the invention, there is a method of protecting a non-cancerous cell from destruction by a cancer cell, comprising the step of delivering to the cancer cell an anti-lipocalin agent, a lipocalin-inhibiting agent, a lipocalin-targeting agent, or an anti-secreted lipocalin agent. In specific embodiments, the non-cancerous cell is a bone marrow cell.

In another embodiment of the invention, there is a method of preventing proliferation of one or more cancer cells in an individual, comprising the step of delivering to the individual an agent that inhibits secretion of lipocalin or targets secreted lipocalin from one or more chronic myeloid leukemia cells. In specific embodiments, the method is further defined as preventing destruction of a non-cancerous cell by secreted lipocalin from one or more chronic myeloid leukemia cells. In additional specific embodiments, the method further comprises the step of treating the individual with an additional cancer therapy.

In another embodiment of the present invention, there is a method of treating an individual with cancer comprising the step of providing to the individual an agent that targets secretion of lipocalin, an agent that targets secreted lipocalin, or an agent that does both. In specific embodiments, an agent that targets secretion of lipocalin targets the expression of lipocalin, such as by reducing its level of expression.

In an additional embodiments, there is a method of protecting a non-cancerous cell from apoptosis, comprising the step of providing to the individual an agent that targets secretion of lipocalin, an agent that targets secreted lipocalin, or an agent that does both.

In specific embodiments, there are two forms of NGAL/24p3. One of these (with 24p3) is the smaller form, in specific embodiments of the invention. With NGAL, cancer patients, such as leukemia (including CML), breast, or prostate cancer patients secrete only one form, and in particular embodiments it is the smaller form.

In another embodiment of the invention, there is an agent that inhibits lipocalin. In specific embodiments, the agent inhibits a 25 kDa form, a 21 kDa form, or both. In specific embodiments, an agent inhibits lipocalin such that the agent prevents action of lipocalin on a non-cancerous cell. In further specific embodiments, the agent inhibits the apoptotic activity of lipocalin. In particular aspects of the invention, the agent binds to a secreted form of lipocalin, whereas in other aspects the agent reduces expression of lipocalin, reduces its secretion, or a combination thereof.

In an embodiment of the present invention, there is a method of inhibiting secretion of lipocalin from a cell or targeting secretion of lipocalin from a cell of an individual, comprising the step of administering to the individual a therapeutically effective amount of a lipocalin-inhibiting substance. In specific embodiments, the lipocalin-inhibiting substance comprises an antibody, such as a monoclonal antibody. In other specific embodiments, the lipocalin-inhibiting substance comprises antisense RNA, siRNA, or both. In particular aspects of the invention, the lipocalin-inhibiting substance is identified by a screening method of the present invention.

In one embodiment of the invention, there is a method of screening for an agent that inhibits secretion of lipocalin from a cell or that targets secreted lipocalin from a cell, comprising the steps of obtaining lipocalin; providing a test compound suspected of binding lipocalin; and assaying for the modulation of lipocalin by said test compound, wherein when said test compound modulates lipocalin, said test compound is said agent. In a specific embodiment, the method further comprises the manufacturing of the agent. In a specific embodiment, the modulation is further defined as the binding of lipocalin by the test compound, and in a further specific embodiment, the modulation of lipocalin by the test compound renders lipocalin biologically inactive.

Exemplary methods of screening may comprise providing a cell that secretes lipocalin into a medium; providing a test compound to the cell, the medium or both; and assaying the medium for the presence of lipocalin, assaying modulation of lipocalin by the test compound, or both. In a specific embodiment, the test compound is an antibody, such as a monoclonal antibody, a small molecule, a nucleic acid, a polypeptide, a peptide, or a mixture thereof. Nucleic acids may comprises antisense RNA, siRNA, or both.

In specific embodiments of the screen, the cell is a leukemia cell, such as a chronic myeloid leukemia cell, or a breast cancer cell or a prostate cancer cell. In further embodiments, a therapeutically effective amount of the agent identified by the method is delivered to an individual, such as one who has cancer, for example leukemia, including chronic myeloid leukemia. In other specific embodiments, the cancer is breast cancer or prostate cancer, for example.

In another embodiment of the invention, the individual treated with an agent identified by a screen of the invention is also treated with an additional cancer therapy, such as chemotherapy, transplant, radiation, surgery, gene therapy, hormone therapy, immunotherapy, or a combination thereof.

In a particular embodiment, there is an agent identified by a screening method of the invention. The agent, which may be referred to as a substance, may be comprised in a pharmaceutically acceptable excipient. The agent may be comprised in a liposome or in a lentiviral vector, for example.

In additional embodiments of the invention, there is a method of protecting a non-cancerous cell from destruction by lipocalin in an individual, comprising the step of delivering to the individual an agent identified by a screening method of the invention. In a specific embodiment, the non-cancerous cell is a bone marrow cell.

In another embodiment of the invention, there is a method of preventing proliferation of one or more cancer cells in an individual, comprising the step of delivering to the individual an agent that inhibits secretion of lipocalin or that targets secreted lipocalin from one or more cancer cells. In a specific embodiment, the method is further defined as preventing destruction of a non-cancerous cell by secreted lipocalin. In a specific embodiment, the cancer cell is a leukemia cell, a breast cancer cell, or a prostate cancer cell. The method may further comprise the step of treating the individual with an additional cancer therapy, such as chemotherapy, transplant, radiation, surgery, gene therapy, hormone therapy, immunotherapy, or a combination thereof. The agent may be identified by an exemplary screening method of the invention.

In an additional embodiment, there is a kit for cancer therapy comprising a composition, said composition housed in a suitable container and comprising an agent identified by a screening method of the invention. The composition may be suitably aliquoted for therapeutic use. The composition may be comprised in a pharmaceutically acceptable excipient. The kit may further comprising an additional cancer therapeutic composition, in specific embodiments of the invention.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows IL-3 independent expression of 24p3 in mouse hematopoietic cell lines expressing P210 BCR-AB (b3a2)L. (a) Expression of 24p3 RNA in mouse hematopoietic cell lines expressing P210 BCR-ABL (b3/a2). RT-PCR was performed to detect 24p3 and actin transcripts in the cell lines listed (b) Expression of BCR-ABL in primary mouse marrow cells stimulates 24p3 expression. Marrow cells were infected with the MigR1 virus encoding either GFP or BCR-ABL(b3a2). 24p3 transcripts were measured by quantitative RTPCR. Transcripts were normalized for loading using GAPDH transcripts. The results are average of three experiments. (c) 24p3 expression is dependent on Bcr-Abl expression. Tet-off BCR-ABL+ 32D cells were maintained in different doses of tetracycline for several days before analysis in the presence of IL-3 to maintain viability and cell proliferation at low levels of BCR-ABL. Total RNA was extracted and the levels of 24p3 and BCR-ABL RNA normalized by GAPDH RNA levels were examined by quantitative Real-time PCR. (d) The tyrosine kinase activity of Bcr-Abl is required for expression of 24p3 transcripts. BCRABL+ 32D cells were treated with 10 μM imatinib for 1 hr to 24 hr. The level of 24p3 RNA normalized by GAPDH RNA levels of treated and untreated cells were determined by quantitative Real-Time PCR. (e) Imatinib does not inhibit induction of 24p3 caused by withdrawal of IL-3 in BCR-ABL+ 32D cells. The experiment was performed as in panel d, except the cells were washed free of IL-3 before treatment with imatinib. (f) CM from BCR-ABL+ 32D cells induces cell death in 32D cells. Annexin V/PI flow cytometry analyses were performed on 32D cells treated with CM from 32D cells (harvested after 72 hr) either deprived of IL-3 or maintained in IL-3 (3 n ml), and CM from 32D cells expressing P210 BCR-ABL maintained in the presence and absence of IL-3. (g) BCR-ABL+ cells are resistant to apoptosis caused by 24p3. Annexin V/PI flow cytometry analyses was performed on P210 BCR-ABL positive 32D cells incubated with CM obtained from 32D cells expressing P210 BCR-ABL maintained in the presence and absence of IL-3. Target cells were treated in wells at about 50,000 cells per ml.

FIG. 2 shows reduction of 24p3 expression in BCR-ABL+ cells by anti-sense 24p3 and 24p3 siRNAs inhibits apoptosis activity of CM. (a), Anti-sense 24p3 expression reduces to expression of 24p3 protein in BCR-ABL+ 32D cells. Top panel, Western blotting with 24p3 antibody, and actin antibody as a loading control. Bottom panel, quantitation of band intensity normalized for loading by actin band intensity. (b) 24p3 siRNA expression reduces the expression of 24p3 protein. Top panel, Western blotting with 24p3 antibody and actin antibody. Bottom panel, quantitation of band intensity normalized for loading by actin. (c) Anti-sense 24p3 expression and 24p3 siRNA in BCR-ABL+ 32D cells reduced the 24p3 protein level in CM. Western blotting of CM was performed with 24p3 antibody. CM was collected from equivalent number of cells maintained 24 hr in the absence of serum. (d) Anti-sense 24p3 expression and 24p3 siRNA expression in BCR-ABL+ 32D cells reduce the level of apoptosis induced in 32D target cells and FL5.12 target cells after exposure to CM for 48 hr in the presence of 3 ng/ml of recombinant IL-3. Controls include target cells maintained with and without 3 ng/ml of IL-3. (e) Co-culture of BCR-ABL+ 32D cells with 32D cells decreases the number of viable 32D cells. GFP negative 32D cells (GFP-32D cells) were co-cultured with either 32D GFP+ cells or BCR-ABL+ 32D GFP cells in a 1:1 ratio in presence of 3 ng/ml recombinant IL-3. The cell cultures were diluted 2-fold with fresh culture medium with IL-3 every 2 days to maintain vigorous growth. The amount of viable GFP negative 32D cells were analyzed on day 0, 3, 7 and 13 by flow cytometry. (f) Antibody to 24p3 blocks the decrease in viability of IL-3-dependent primary bone marrow cells caused by conditioned medium from BCR-ABL+ 32D cells. CM from BCRABL+32D cells was supplemented with IL-3 (2 ng/ml) and added to BALB/c primary bone marrow cells. 1.5 μg of affinity-purified rabbit antibody to 24p35 or 2 μg of pre-immune rabbit serum was added at the same time. Cell viability was determined by trypan blue staining after 48 hrs.

FIG. 3 shows anti-sense 24p3 studies. (a) Anti-sense 24p3 expression in BCR-ABL+ 32D cells extends survival in the NOD/scid mouse model. The * indicates death due to leukemia; other mice were sacrificed because of severe illness to allow evaluation of the tissue pathology and GFP content. b, Photomicrograph of an H&E stained spleen section of NOD/scid mice given with 32D cells. The M refers to megakaryocyte. The lower panel is an enlargement of the area outlined in the upper panel (c) Expression of anti-sense 24p3 in BCR-ABL+ 32 D cells permits normal hematopoiesis in the spleen of leukemic mice. Erythroid hematopoiesis is apparent due to the presence of small darkly stained cells in photomicrograph of an H&E stained spleen section of NOD/scid mice injected with BCR-ABL+32D cells expressing anti-sense 24p3. The M refers to the large megakaryocytes indicative of active hematopoiesis; T refers to tumor cells. The lower panel is an enlargement of the area outlined in the upper panel. (d) BCR-ABL+ 32D cells transduced with the GFP lentivirus GFP vector have reduced levels of normal hematopoiesis. H &E stained spleen section of NOD/scid mice given with BCRABL+32D cells expressing GFP vector. The T refers to tumor cells. The lower panel is an enlargement of the area outlined in the upper panel. (e) An H&E stained bone marrow section of NOD/scid mice given 32D/GFP cells. The M refers to a megakaryocyte. The lower panel is an enlargement of the area outlined in the upper panel. (f) Expression of anti-sense 24p3 in BCR-ABL+ 32 D cells permits normal hematopoiesis in the marrow of leukemic mice. An H&E stained bone marrow section of NOD/scid mice given with BCR-ABL+ 32D cells expressing anti-sense 24p3. The M refers to nucleated erythroid cells and megakaryocytes. The lower panel is an enlargement of the area outlined in the upper panel. (g), BCR-ABL+ 32D cells transduced with the a lentivirus GFP vector has reduced levels of normal hematopoiesis. H&E stained bone marrow section of NOD/scid mice given with BCR-ABL+ 32D cells expressing GFP vector. The T refers to tumor cells. The lower panel is an enlargement of the area outlined in the upper panel.

FIG. 4 shows additional anti-sense 24p3 studies. (a) Expression of anti-sense 24p3 strongly reduced level of engraftment of BCR-ABL+ 32D cells in spleen (left) and marrow (right) of leukemic mice. NOD/scid mice were irradiated sub-lethally and mice were injected i.v. with 10e6 BCR-ABL+ 32D cells either expressing GFP only (Vector) or anti-sense (AS) 24p3 and GFP. Control mice were irradiated and received no leukemia cells (IRR). At 16 to 18 days after injection mice were sacrificed and spleen and bone marrow (BM) cells were extracted; mature red cells were removed from the cell suspension using RBC lysis buffer. Engrafted leukemia cells (GFP+ cells) in spleen and marrow samples were measured by Flow cytometry. (b) Reduction of secretion of 24p3 by anti-sense 24p3 expressing BCR-ABL+ 32D cells in spleen and marrow tissues of leukemic mice. Supernatant fluid from spleen and marrow tissue was harvested from leukemic mice at day 18 after challenge with BCR-ABL+ 32D cells. The supernatant fluid was concentrated and analyzed by Western blotting with anti-24p3 on a 15% SDS polyacrylamide gel. Purified recombinant 24p3 (r24p3) was used as a positive control of Western Blotting using anti-24p3 antibody. Equal amounts of protein from various fractions was applied to the gel. IRR represents tissue fractions from control irradiated mice not challenged with BCR-ABL+ 32D cells. The bottom histogram shows the quantitation of the 24p3 proteins shown in the upper panel. (c) Anti-sense 24p3 expression in BCR-ABL+ 32D cells restores platelet levels to normal ranges in NOD/scid and C3H/HeJ mice. Platelet levels in peripheral blood (PB) of NOD/scid and C3H/HeJ mice injected with either BCR-ABL+ 32D cells expressing anti-sense 24P3 (AS), Sense 24P3 (Sense), or GFP/Vector control (Vector) were determined by the staff in the Veterinary Hematology Lab. (d-i) Stimulation of normal hematopoiesis in leukemic mice by reduction of 24p3 expression in BCR-ABL+ 32D cells. NOD/scid mice were processed as in a. The GFP-negative spleen and marrow cells were stained with a myeloid marker (anti-Mac-1 PerCP-Cy5.5) and the erythroid marker (anti-TER119 APC). Cells were gated to exclude GFP+ cells to characterize the normal hematopoietic cells. Non-erythroid/myeloid GFP negative cells were also measured; in NOD/scid mice, these were predominantly stromal cells. The flow analysis values of cells from tissues were obtained from 3-5 mice at each time point for each group (e.g. IRR, AS, Vector). Values shown are an average of 3-5 values.

FIG. 5 shows that anti-sense 24p3 expression increases the survival of mouse hematopoietic cells deprived of IL-3. FL512 cells transduced with anti-sense 24p3 (AS), sense 24p3 (S) or a vector control (Vector) were deprived with IL-3. After 48 hours, the level of apoptotic cells were measured by flow cytometry using Annexin V staining.

FIG. 6 demonstrates that expression of anti-sense or siRNA 24p3 does not change the Bcr-Abl protein levels. BCR-ABL+ 32D cells expressing vector only, anti-sense 24p3, two different siRNAs against 24p3 were harvested to perform Western blotting. Equal amounts of proteins were loaded in each lane. Bcr-Abl protein levels were detected by using anti-Abi monoclonal antibody 8E9 and anti-actin was used for loading control. The lower graph shows the Bcr-Abl protein levels normalized for the corresponding actin levels.

FIGS. 7a-7f show that the 21 kDa form of NGA1 is the form secreted by BCR-ABL. In FIG. 7a, the 21 kDa form of NGAL is the major form secreted by BCR-ABL+CML cells. Marrow supernatant fluid from CML patients contains both the 25 kDa and 21 kDa forms of NGAL but the 21 kDa form of NGAL is specifically secreted by BCR-ABL+CML marrow cells. Marrow supernatant fluids were harvested from four chronic phase CML patients with differing levels of BCR-ABL+ cells per marrow sample (as determined by quantitative Real-time RT-PCR). An equal amount of protein (1.5 mg) was loaded on each lane of a 3.0 mm thick polyacrylamide gel after complete denaturing by boiling in SDS/mercaptoethanol sample buffer. The samples were analyzed by Western blotting with commercial NGAL antibody. b, CM from NGAL-transfected COS1 cells induces apoptosis in mouse primary bone marrow cells. Similar results were obtained with MT4 (human T cells). FIGS. 7c and 7d, Soft agar clones of K562 cells express higher levels of NGAL transcripts compared to uncloned K562 cells. Transcripts were measured by RT-PCR (FIG. 7c) and quantitative Real-time RT-PCR (FIG. 7d). In FIG. 7e, NOD/scid mice injected with K562 cells expressing high levels of NGAL have shorter survival than mice injected with uncloned K562 cells. In FIG. 7f, Spleen and marrow tissues from mice injected with high NGAL-expressing clones of K562 cells have severe suppression of normal hematopoiesis and no evidence of tumor formation. The c5 spleen and marrow are from mouse #4 (terminally ill at 34 days post challenge with clone 5 K562 leukemia cells). Magnification-250. More severe suppression of hematopoiesis was seen in mouse # 1 who was terminally ill at day 13. These tissues have the same properties as tissues that were identified to have “atrophy” of mouse hematopoiesis in a previous paper of the inventors.

FIG. 8 provides co-culture studies of the invention. In FIG. 8a, there is co-culture of BCR-ABL+ 32D cells with 32D cells without barrier decreases the number of 32D cells due to induction of apoptosis. GFP negative 32D cells (GFP-32D cells) were co-cultured with either 32D GFP+ cells or BCR-ABL+ 32D GFP cells in a 1:1 ratio in presence of 3 ng/ml recombinant IL-3. The cell cultures were diluted 2-fold with fresh culture medium with IL-3 every 2 days to maintain vigorous growth. The amount of viable GFP negative 32D cells were analyzed on day 0, 3, 7 and 13 by flow cytometry to determine those cells that were negative for GFP and Annexin V staining. b. Co-culture of BCR-ABL+ 32D cells with 32D cells in a culture dish with a barrier induces apoptosis of 32D cells. GFP-negative 32D cells were co-cultured with 32D GFP+ cells or BCR-ABL+ 32D GFP cells in a culture dish with a barrier to prevent cell mixing. All cells were grown in the culture medium with 3 ng/ml and diluted 2 fold every 2 days. Annexin V staining of GFP-negative 32D cells was determined by flow cytometry on days 3, 7 and 13.

FIG. 9 demonstrates NGAL expression in human prostate cancer cell lines. A. RT-PCR analyses of NGAL transcripts in different kinds of prostate cancer cells. RNA was extracted and same amount of RNA was used in the reverse transcriptase reaction to make cDNA. RT-PCR was performed by using same amount of cDNA. B. Western blotting analyses of NGAL expression in prostate cancer cells. Cell lysates from three different human prostate cell lines were examined by Western blotting with anti-NGAL. Conditioned Medium (CM) from NGAL transfected CosI cells was used as a positive control for the 24 kDa form of NGAL. Actin was used as a loading control for the cell lysate samples. These results indicate that PC3 prostate cancer cells, known to invade bone in an animal model, expressed high levels of NGAL. In contrast LNCap and DU145 express very little or no NGAL protein. These latter cells do not invade bone.

FIG. 10 shows NGAL expression in human prostate cancer cell lines. A. RT-PCR analyses of NGAL transcripts in different kinds of prostate cancer cells. RNA was extracted and same amount of RNA was used in the reverse transcriptase reaction to make cDNA. RT-PCR was performed by using same amount of cDNA. B. Western blotting analyses of NGAL expression in prostate cancer cells. Cell lysates from three different human prostate cell lines were examined by Western blotting with anti-NGAL. Conditioned Medium (CM) from NGAL transfected Cos1 cells was used as a positive control for the 24 kDa form of NGAL. Actin was used as a loading control for the cell lysate samples. These results indicate that PC3 prostate cancer cells, known to invade bone in an animal model, expressed high levels of NGAL. In contrast LNCap and DU145 express very little or no NGAL protein. These latter cells do not invade bone.

FIGS. 11A-11G concern NGAL expression associated with a variety of parameters. Breast tumor specimens were examined for expression of NGAL (FIG. 11A). FIG. 11B shows NGAL expression in breast cancer tissues by microarray. FIG. 11C provides a correlation of ER status and NGAL expression. FIG. 1D shows correlation of HER-2 and NGAL expression. FIG. 11E provides correlation of NGAL and Tumor size. FIGS. 11F and 11G show relationship of NGAL expression to BMN grades.

FIGS. 12A-12B concern different cell lines and their association with NGAL expression. FIG. 12A shows that the MCF-7 cell line [HER-2 (−), ER(+)] had lower levels of secreted NGAL compared to a HER2/neu+ SKBr3 cell line. FIG. 12B demonstrates treatment of SKBr3 cells with Herceptin, the anti-HER2 antibody, which inhibited NGAL protein expression.

FIG. 13 shows that NGAL expression is down-regulated by the exemplary PI3K Inhibitor LY 294002.

FIG. 14 demonstrates that the Akt pathway is required for NGAL expression. FIG. 15 illustrates that NFkB inhibition with the exemplary Bay 11-7082 compound attenuates NGAL expression.

FIG. 15 shows NGAL expression in conditioned medium having been in the presence of particular compounds.

FIG. 16 illustrates an exemplary model of NGAL expression in breast cancer.

FIG. 17 shows characteristics of the exemplary cell lines employed herein.

FIGS. 18A-18B concern detection of two forms of NGAL. FIG. 18A shows detection of the two forms of NGAL in conditioned medium (CM) of COS-1 cells transfected with NGAL or conditioned medium of PC-3 cells (FIG. 18B).

FIG. 19 shows a high rate of apoptosis as detected by Annexin V staining, which was observed in exemplary hematopoietic 32D cells cultured with CM derived from exemplary PC-3 cells.

FIG. 20A shows knocking down of NGAL level using exemplary RNAi oligonucleotides (#3) and (#4) lowered the cell death inducing activity in CM of PC-3 cells. FIG. 20B illustrates % relative death in PC-3 cells transfected with either of the two exemplary RNAi oligonucleotides.

FIG. 21 illustrates expression of 24p3/NGAL in multiple tumor cell lines, including at least PC-3 cancer cells and 4T-1 breast cancer cells and which demonstrates that there are at least two forms of 24p3/NGAL therein.

FIG. 22 shows induction of apoptosis using conditioned media from the exemplary tumor cell lines.

FIG. 23 killing activity using CM from 24p3/NGAL-His transfected cells is demonstrated.

FIG. 24 demonstrates glycosylation studies on both forms of 24p3/NGAL.

FIGS. 25A-25E demonstrate multiple experiments regarding 24p3 expression.

FIG. 26 shows that soft agar clones of K562 cells that express high levels of NGAL suppress hematopoiesis.

FIG. 27 illustrates an exemplary model for NGAL involvement in marrow expansion of leukemia cells. The model also applies in breast and prostate cells. Ph+relates to Philadelphia chromosome (abnormal 22) that encodes the BCR-ABL leukemia gene seen in most CML patients.

FIG. 28 provides rNGAL experiments from plasma from CML patients versus normal individuals.

### DETAILED DESCRIPTION OF THE INVENTION

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The term “biologically inactive” as used herein refers to lipocalin having at least reduced if not completely inhibited capability of inducing apoptosis in a cell to which it targets. In other words, the term refers to lipocalin having reduced or completely inhibited pro-apoptotic activity compared to wild type.

The term “chronic myeloid leukemia,” which may be referred to as CML, is characterized by leukemic myeloid progenitors that overpopulate the bone marrow and exit the bone marrow prematurely, invading the spleen and liver. In specific embodiments of the invention, it is caused by the Bcr-Abl oncoprotein, which results from the formation of the Philadelphia (Ph) chromosome in pluripotent stem cells in bone marrow.

The term “hematopoiesis” as used herein refers to the process of replacing normal blood cells that normally die in the course of their lifespan.

The term “leukemia” as used herein refers to cancer of blood-forming organs (such as bone marrow cells) and that may result in the uncontrolled production of abnormal leukocytes (white blood cells).

The term “modified” as used herein refers to a form of lipocalin that is different from wild-type lipocalin 2 gene product. In specific embodiments, the modified form is detectably different, such that discrimination can be made between lipocalin secreted from normal cells and lipocalin that is secreted from a cancer cell and that has cell death-inducing activity against a non-cancerous cell, such as a normal hematopoietic cell. Any modification of lipocalin is encompassed by the invention, although in particular embodiments the modification results from a transcriptional event, such as by alternative splicing, for example, or a post-translational change, such as a change in phosphorylation status, for example. The modified form may comprise a mutation that affects the biological activity of lipocalin gene product, including its apoptotic activity against a non-cancerous cell, for example.

The terms “targeting” or “targeted” as used herein refers to binding of an agent or test compound to lipocalin or targeting the expression thereof such that it renders lipocalin reduced in level, biologically inactive, or both. In alternative embodiments, targeting includes binding of an agent or test compound to a molecule that acts in conjunction with lipocalin such that it renders lipocalin reduced in level, biologically inactive, or both.

### II. The Present Invention

The present invention concerns targeting a cell death molecule, such as lipocalin, and/or its secretion from a cancerous cell. In particular embodiments, this targeting prevents lipocalin from deleteriously affecting another cell, such as a non-cancerous cell. In certain aspects, lipocalin deleteriously affects the non-cancerous cell through binding of a cell surface receptor, for example. Although any cancerous cell may secrete lipocalin, in specific embodiments, the invention concerns secretion of lipocalin from a leukemia cell.

In specific embodiments, there is suppression of normal hematopoiesis by lipocalin 2 secreted by BCR-ABL positive hematopoietic cells. In particular aspects of the invention, the tyrosine kinase activity of Bcr-Abl is required for expression of 24p3 transcripts.

Treatment results for various leukemias have improved significantly, with an increasing number of patients having long-term, disease-free survival. Nevertheless, various patient groups still fare poorly with current approaches. The atrophy of normal tissues in different organs results in malfunction of important organs. A lot of individuals even lose chances of receiving potentially curative therapy because of poor functioning organs. Nowadays due to poor understanding of the mechanism of atrophy in leukemia, the therapies mainly focus on a syndrome supporting, such as using GM-CSF, G-CSF or EPO to stimulate bone marrow cell growth. The present invention provides a novel way to improve the atrophy based on the mechanism identified by the present inventors.

The invention concerns a new discovery relating to the disease properties of leukemia. CML cells secrete a cell death factor that causes death of normal blood cells while the leukemia cells themselves are resistant to this factor. It is a common phenomenon that a majority of kinds of different leukemia cells can causes aplasia in normal cells. The present invention elucidates at least part of the mechanism of atrophy of normal bone marrow or other tissues in leukemia disease and identifies treatment against aplasia, thereby alleviating a patient's anemia and other wasting syndromes.

In specific aspects of the invention, BCR-ABL positive mouse myeloid 32D cells secrete 24p3. In specific embodiments of the invention, the present inventors have obtained interesting new data regarding a possible Bcr-Abl mechanism for induction of cell death within normal mouse hematopoietic cells. Michael Green and colleagues have described a gene product, 24p3, a lipocalin, which is secreted by mouse hematopoietic cells that are deprived of Il1-3. This factor induced apoptosis in normal blood cells and IL-3 dependent cell lines. The present inventors have obtained the cDNA clone of 24˜3 and its sequence from Dr. Green and found that Bcr-Abl expressing 32D cells also express 24p3 RNA as measured by RT-PCR. Moreover these Bcr-Abl 32D cells are resistant to the apoptotic effects of 24p3. The findings indicate that the cell death effects of conditioned medium from 24p3 expressing cells can be reversed by IL-3 in a dose-dependent manner. However, Bcr-Abl expressing cells are very resistant to the cell death effects of 24p3 compared to normal cells despite producing 24p3 RfA. The conditioned medium is collected from 24P3 antisense-treated cells and assayed.

### III. Lipocalin

Lipocalins bind to small, hydrophobic molecules such as retinol, or binds to specific cell-surface receptors or forms macro-molecular complexes. Lipocalins have a diverse primary sequence (20% homology) with three conserved short motifs, yet there is a similar tetrary structural: a single eight-stranded continuously hydrogen-bonded antiparallel β-barrel

Diverse functions include retinol transport; cryptic coloration; olfaction; pheromone transport; prostaglandin synthesis; regulation of the immune response; cell homeostatic mediation; and iron transportation and acquisition. Lipocalin 2 is induced in IL-3 dependent hematopoietic cells when deprived of IL-3 (Devireddy et al., 2001).

An exemplary nucleotide sequence of lipocalin (lipocalin 2 (oncogene 24p3)) is comprised in GenBank Accession No. NM_{—}005564 (SEQ ID NO:8) which encodes the exemplary polypeptide of GenBank Accession No. NP_{—}005555 (SEQ ID NO:9). A skilled artisan recognizes that similar sequences are available for lipocalin sequences at the National Center for Biotechnology Information's GenBank database. Lipocalin may be referred to as LCN2 or NGAL, such as in humans, for example. NGAL is found in neutrophils, bone marrow cells, and ovarian cancer cells, and it forms heterodimers with a gelatinase. Human NGAL and murine 24p3 have 71% sequence similarity.

In some embodiments of the invention, the lipocalin comprises a modified polypeptide, for example wherein the polypeptide is glycosylated, or such as lacking particular protein regions from incorrect exon-intron splicing, and so forth.

24p3/NGAL

The particular lipocalin 24p3/NGAL has homologs in mouse 24p3 (200aa), rat (200aa) and human NGAL (198aa). NGAL is on chromosome 9, with 7 exons, and there is a signal peptide (20aa) for secretion at N-terminus and the N-terminal Q is puroglutamated. The C-terminal is carboxymethylated, and the polypeptide exists in various forms, including as a monomer, homodimer, and heterodimer.

Crystallography studies of 24p3/NGAL indicate that 24p3/NGAL binds to siderophores (iron chelating molecules). Thus, identification of agents that would bind to, and in specific embodiments, inhibit the activity and/or secretion of, include those that can mimic this interaction, for example.

In specific aspects of the invention, 24p3/NGAL exists in a large complex of molecules, and in further specific embodiments, conserved cysteines link 24p3/NGAL to other proteins. In further specific embodiments, the 21 kDa form of NGAL/24p3 is active in the induction of apoptosis, whereas in alternative embodiments the 24 kDa form of NGAL/24p3 is active in the induction of apoptosis, or both the 21 kDa and 24 kDa forms of NGAL/24p3 are active in the induction of apoptosis.

A skilled artisan recognizes based on the ample disclosure provided herein that normal marrow cells secrete the 24 kDa form of NGAL, whereas CML marrow cells secrete the 21 kDa form (which may be termed the modified form of NGAL) and the 24 kDa form. Thus, in particular embodiments, the modified form is the apoptotic form.

In particular embodiments of the invention, neutrophil gelatinase-associated lipocalin (NGAL) is highly associated with HER2+/ER— breast cancers and is a downstream effector of PI3K/AKT pathway. The polynucleotide is located at 9q34, and it is a small secreted glyco-protein from lipocalin family.

There is significant correlation between NGAL expression in breast cancer with several markers of poor prognosis, including estrogen and progesterone receptor-negative status and high proliferation (S-phase fraction) (Stoesz et al., 1998). Also, NGAL expression significantly stimulates tumor growth in vivo in a dose-dependent fashion (Fernandez et al., 2005).

### IV. Exemplary Modulators that Target Lipocalin Secretion and/or Activity

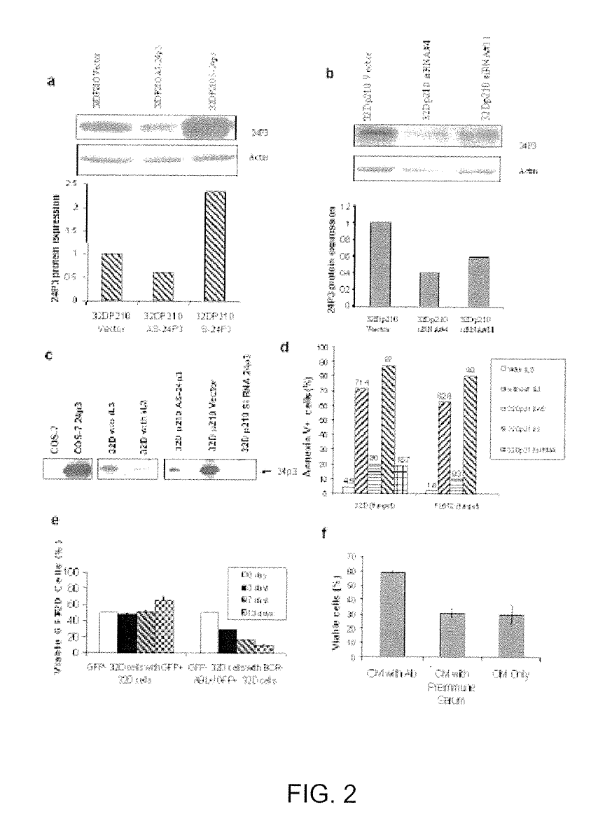

In specific aspects of the invention, there are modulators, which may be referred to as agents, that target the lipocalin secretion and/or its activity. The modulators may be of any suitable type such that they inhibit the ability of lipocalin from a cancer cell to negatively affect a non-cancerous cell. In particular embodiments, the modulator may be an antibody, a small molecule, a polynucleotide, a polypeptide, a peptide, or a combination or mixture thereof, for example. In specific embodiments of the invention, anti-sense 24p3 and 24p3 siRNA expression reduces expression of 24p3 protein in BCR-ABL+ 32D cells (FIG. 2). In further specific embodiments, anti-sense and siRNA 24p3 expression reduce the 21 kDa form of 24p3 in conditioned media (CM). In specific embodiments, this reduction in expression leads to a reduced amount of available lipocalin, such as a reduced amount of secreted lipocalin, both of which result in a reduced amount available to deleteriously affect non-cancerous cells. In specific embodiments, this results in reduction of the level of apoptosis in normal cells.

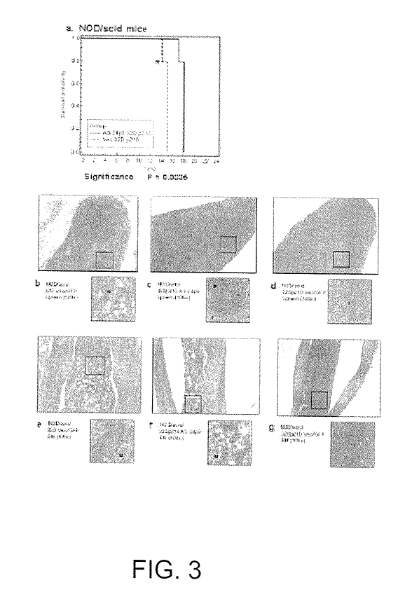

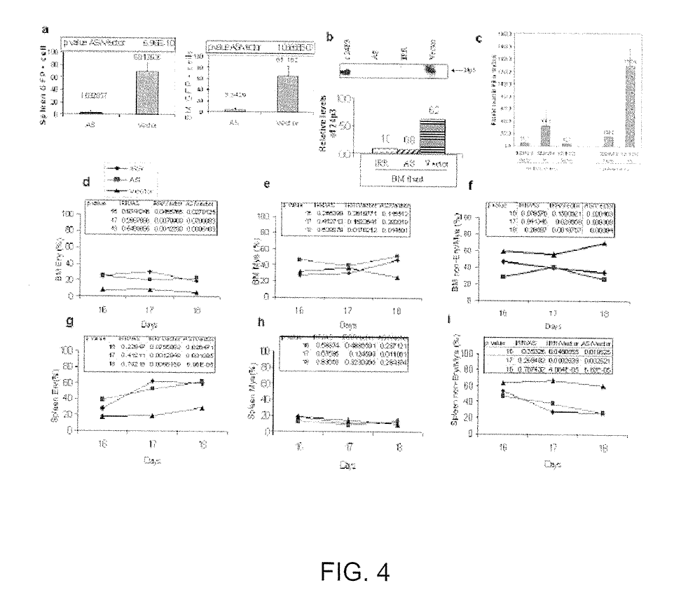

In other embodiments of the invention, antibodies to 24p3 block apoptosis in IL-3-dependent primary bone marrow cells by conditioned medium from BCR-ABL+32D cells. (FIG. 2) This in turn leads to stimulation of normal hematopoiesis in leukemic mice by reduction of 24p3 expression in BCR-ABL+ 32D cells (FIGS. 3 and 4).

### V. Screening for Modulators of Lipocalin Secretion and/or Activity

A skilled artisan recognizes, based on the examples and teachings provided herein, that methods and compositions are useful upon target identification of an agent that modulates lipocalin secretion, lipocalin activity, or both. In a specific embodiment, the present invention further comprises methods for identifying modulators of the function and/or secretion of lipocalin. These assays may comprise random screening of large libraries of candidate substances; alternatively, the assays may be used to focus on particular classes of compounds selected with an eye towards structural attributes that are believed to make them more likely to modulate lipocalin secretion and/or function.

In particular embodiments of the invention, a function of lipocalin is as a pro-apoptotic factor. The present invention in some embodiments targets this activity.

By function, it may be meant that one may assay for the activity for lipocalin to indirectly or directly effect apoptosis on a cell, such as a non-cancerous cell.

To identify a modulator, one generally will determine the activity and/or secretion of lipocalin in the presence and absence of the candidate substance, a modulator defined as any substance that alters function. For example, a method generally comprises:

(a) providing a candidate modulator;

(b) admixing the candidate modulator with an isolated compound or cell, or a suitable experimental animal;

(c) measuring one or more characteristics of the compound, cell or animal in step (b); and

(d) comparing the characteristic measured in step (c) with the characteristic of the compound, cell or animal in the absence of said candidate modulator,

wherein a difference between the measured characteristics indicates that said candidate modulator is, indeed, a modulator of the compound, cell or animal.

Assays may be conducted in cell free systems, in isolated cells, or in organisms including transgenic animals, for example.

It will, of course, be understood that all the screening methods of the present invention are useful in themselves notwithstanding the fact that effective candidates may not be found. The invention provides methods for screening for such candidates, not solely methods of finding them. Furthermore, a skilled artisan recognizes that any molecule which, analogous to lipocalin, causes apoptosis of a non-cancerous cell may be tested in a similar fashion.

A. Modulators

As used herein the term “candidate substance,” “test compound” or “agent” refers to any molecule that may potentially inhibit or enhance lipocalin secretion and/or activity. The candidate substance may be a protein or fragment thereof, a small molecule, an antibody, or even a nucleic acid molecule, for example. It may prove to be the case that the most useful pharmacological compounds will be compounds that are structurally related to glycoprotein binding molecules. Using lead compounds to help develop improved compounds is known as “rational drug design” and includes not only comparisons with known inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one that exerts its inhibitory or activating effect upstream, downstream or directly on lipocalin. Regardless of the type of inhibitor or activator identified by the present screening methods, the effect of the inhibition or activator by such a compound results in affecting lipocalin activity and/or secretion as compared to that observed in the absence of the added candidate substance.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

An exemplary technique for high throughput screening of compounds is described in WO 84/03564. Large numbers of small peptide test compounds are synthesized on a solid substrate, such as plastic pins or some other surface. Bound polypeptide is detected by various methods.

C. In Cyto Assays

The present invention also contemplates the screening of compounds for their ability to modulate lipocalin expression in cells or secretion from cells. Various cell lines can be utilized for such screening assays, including cells specifically engineered for this purpose, such as cancer cells, or cells obtained from a murine cancer model, for example. For example, a cell may preferably comprise a construct comprising a lipocalin sequence operably linked to a reporter sequence. Assessment of a screened compound for affecting lipocalin expression is based upon the effect it has on reporter sequence expression.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., pro-apoptotic compound, enzyme, receptor, hormone) or cell (e.g., growth, tumorigenicity, survival), or instead a broader indication such as behavior, anemia, immune response, etc.

The present invention provides methods of screening for a candidate substance that interferes with lipocalin function and/or secretion from a cell. In these embodiments, the present invention is directed to a method for determining the ability of a candidate substance to target lipocalin, generally including the steps of: administering a candidate substance to the animal; and determining the ability of the candidate substance to bind to lipocalin.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### VI. Cancer Therapy

The present invention targets secreted lipocalin and/or the secretion thereof from a cancer cell, such as a leukemia cell, including a chronic myeloid leukemia cell. In particular embodiments, the successful inhibition of activity of lipocalin and/or the successful prevention of secretion of lipocalin or reduction of the amount of secreted lipocalin prevents destruction (such as by apoptosis) of a normal cell or prevents further destruction of normal cells. In specific embodiments of the invention, the targeting of lipocalin protects neighboring normal cells, such as normal cells of the same tissue or organ, but it is advantageous to also treat the cancer cells themselves that are secreting the lipocalin.

A wide variety of cancer therapies, known to one of skill in the art, may be used in combination with the methods or compositions contemplated for the present invention. The inventors can use any of the treatments described herein in addition to administering to a cancer cell. A skilled artisan recognizes that the nature of the additional cancer therapy or therapies correlates with the type of cancer, and therefore will modify the treatment based on the needs of the primary cancer.

A. Radiotherapeutic Agents

Radiotherapeutic agents and factors include radiation and waves that induce DNA damage for example, γ-irradiation, X rays, UV irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X rays range from daily doses of 50 to 200 roentgens for prolonged periods of time (3 to 4 weeks), to single doses of 2000 to 6000 roentgens. Dosage ranges for radioisotopes vary widely, and depend on the half life of the isotope, the strength and type of radiation emitted, and the uptake by the neoplastic cells.

B. Surgery

Surgical treatment for removal of the cancerous growth is generally a standard procedure for the treatment of tumors and cancers. This attempts to remove the entire cancerous growth. However, surgery is generally combined with chemotherapy and/or radiotherapy to ensure the destruction of any remaining neoplastic or malignant cells. Thus, surgery or sham surgery may be used in the model in the context of the present invention.

C. Chemotherapeutic Agents

These can be, for example, agents that directly cross-link DNA, agents that intercalate into DNA, and agents that lead to chromosomal and mitotic aberrations by affecting nucleic acid synthesis. In specific embodiments, the chemotherapy agent delivered to the individual is tailored for the nature of the cancer itself. For example, for individuals with leukemia, Gleevec, interferon, busulfan, or mixtures or combinations thereof and the like may be administered.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

### VII. Pharmaceutical Preparations

Pharmaceutical compositions of the present invention comprise an effective amount of one or more agents that target lipocalin or the secretion thereof or additional agent dissolved or dispersed in a pharmaceutically acceptable carrier. The phrases “pharmaceutical,” “pharmaceutically acceptable,” or “pharmacologically acceptable” refers to molecular entities and compositions that do not produce an adverse, allergic or other untoward reaction when administered to an animal, such as, for example, a human, as appropriate. The preparation of a pharmaceutical composition that contains at least one agent that targets lipocalin or the secretion thereof and/or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the composition is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations that are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### VIII. Immunological Reagents

In particular embodiments of the invention, immunological reagents are employed. For example, antibodies may be utilized to bind lipocalin, thereby rendering the lipocalin molecule at least partially ineffective for effecting a non-cancerous cell. In other embodiments, antibodies to lipocalin are employed in diagnostic aspects of the invention, such as for detecting the presence of lipocalin secreted from a cell. The antibodies may be of any suitable kind, although in particular embodiments they comprise humanized monoclonal antibodies, for example.

A. Antibodies

In certain aspects of the invention, one or more antibodies may be produced to the expressed lipocalin, the secreted lipocalin, or both. These antibodies may be used in various diagnostic or therapeutic applications described herein.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

“Mini-antibodies” or “minibodies” are also contemplated for use with the present invention. Minibodies are sFv polypeptide chains which include oligomerization domains at their C-termini, separated from the sFv by a hinge region. Pack et al. (1992) Biochem 31:1579-1584. The oligomerization domain comprises self-associating alpha.-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J Immunology 149B:120-126.

Antibody-like binding peptidomimetics are also contemplated in the present invention. Liu et al. Cell Mol Biol (Noisy-le-grand). 2003 March; 49(2):209-16 describe “antibody like binding peptidomimetics” (ABiPs), which are peptides that act as pared-down antibodies and have certain advantages of longer serum half-life as well as less cumbersome synthesis methods.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. A “humanized antibody” is an antibody comprising a humanized light chain and a humanized heavy chain immunoglobulin.

B. Methods for Generating Monoclonal Antibodies

The methods for generating monoclonal antibodies (MAbs) generally begin along the same lines as those for preparing polyclonal antibodies. Briefly, a polyclonal antibody is prepared by immunizing an animal with a LEE or CEE composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art. Antibodies of the invention can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia, synthetic compositions or LEEs or CEEs encoding such adjuvants.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m2) (Johnson/Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60 61), but mice are preferred, with the BALB/c mouse being most preferred as this is most routinely used and generally gives a higher percentage of stable fusions.

The animals are injected with antigen, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody producing B lymphocytes from the immunized animal are then fused with cells of an immortal myeloma cell, generally one of the same species as the animal that was immunized. Myeloma cell lines suited for use in hybridoma producing fusion procedures preferably are non antibody producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65 66, 1986; Campbell, pp. 75 83, 1984). cites). For example, where the immunized animal is a mouse, one may use P3 X63/Ag8, X63 Ag8.653, NS1/1.Ag 4 1, Sp210 Ag14, FO, NSO/U, MPC 11, MPC11 X45 GTG 1.7 and S194/5XX0 Bul; for rats, one may use R210.RCY3, Y3 Ag 1.2.3, IR983F and 4B210; and U 266, GM1500 GRG2, LICR LON HMy2 and UC729 6 are all useful in connection with human cell fusions. See Yoo et al., J Immunol Methods. 2002 Mar. 1; 261(1-2): 1-20, for a discussion of myeloma expression systems.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8 azaguanine resistant mouse murine myeloma SP2/0 non producer cell line.

Methods for generating hybrids of antibody producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 7174, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

Further, expression of antibodies of the invention (or other moieties therefrom) from production cell lines can be enhanced using a number of known techniques. For example, the glutamine synthetase and DHFR gene expression systems are common approaches for enhancing expression under certain conditions. High expressing cell clones can be identified using conventional techniques, such as limited dilution cloning and Microdrop technology. The GS system is discussed in whole or part in connection with European Patent Nos. 0 216 846, 0 256 055, and 0 323 997 and European Patent Application No. 89303964.4.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 104 times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Another embodiment of the invention for producing antibodies according to the present invention is found in U.S. Pat. No. 6,091,001, which describes methods to produce a cell expressing an antibody from a genomic sequence of the cell comprising a modified immunoglobulin locus using Cre-mediated site-specific recombination is disclosed. The method involves first transfecting an antibody-producing cell with a homology-targeting vector comprising a lox site and a targeting sequence homologous to a first DNA sequence adjacent to the region of the immunoglobulin loci of the genomic sequence which is to be converted to a modified region, so the first lox site is inserted into the genomic sequence via site-specific homologous recombination. Then the cell is transfected with a lox-targeting vector comprising a second lox site suitable for Cre-mediated recombination with the integrated lox site and a modifying sequence to convert the region of the immunoglobulin loci to the modified region. This conversion is performed by interacting the lox sites with Cre in vivo, so that the modifying sequence inserts into the genomic sequence via Cre-mediated site-specific recombination of the lox sites.

Alternatively, monoclonal antibody fragments encompassed by the present invention can be synthesized using an automated peptide synthesizer, or by expression of full-length gene or of gene fragments in E. coli.

C. Antibody Conjugates

The present invention further provides antibodies against lipocalin proteins, polypeptides and peptides, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvenmann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti cellular agent, and may be termed “immunotoxins?”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine211, 14carbon, 51chromium, 36chlorine, 57cobalt, 58cobalt, copper67, 152Eu, gallium67, 3hydrogen, iodine123, iodine[25, iodine[31, indium111, 59iron, 32phosphorus, rhenium186, rhenium188, 75selenium, 35sulphur, technicium99m and/or yttrium90. 125I is often being preferred for use in certain embodiments, and technicium99m and/or indium111 are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium99m by ligand exchange process, for example, by reducing pertechnate with stanous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In another embodiment of the invention, the anti-lipocalin antibodies are linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

D. Immunodetection Methods

In still further embodiments, the present invention concerns immunodetection methods for binding, purifying, removing, quantifying and/or otherwise generally detecting biological components such as lipocalin protein components. The lipocalin antibodies prepared in accordance with the present invention may be employed to detect wild type and/or mutant lipocalin proteins, polypeptides and/or peptides. As described throughout the present application, the use of wild-type and/or mutant lipocalin specific antibodies is contemplated. Some immunodetection methods include enzyme linked immunosorbent assay (E LISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle MH and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing lipocalin protein, polypeptide and/or peptide, and contacting the sample with a first anti-lipocalin antibody in accordance with the present invention, as the case may be, under conditions effective to allow the formation of immunocomplexes.

These methods include methods for purifying wild type and/or mutant lipocalin proteins, polypeptides and/or peptides as may be employed in purifying wild type and/or mutant lipocalin proteins, polypeptides and/or peptides from patients' samples and/or for purifying recombinantly expressed wild type or mutant lipocalin proteins, polypeptides and/or peptides. In these instances, the antibody removes the antigenic wild type and/or mutant lipocalin protein, polypeptide and/or peptide component from a sample. The antibody will preferably be linked to a solid support, such as in the form of a column matrix, and the sample suspected of containing the wild type or mutant lipocalin protein antigenic component will be applied to the immobilized antibody. The unwanted components will be washed from the column, leaving the antigen immunocomplexed to the immobilized antibody, which wild type or mutant lipocalin protein antigen is then collected by removing the wild type or mutant lipocalin protein and/or peptide from the column.

The immunobinding methods also include methods for detecting and quantifying the amount of a wild type or mutant lipocalin protein reactive component in a sample and the detection and quantification of any immune complexes formed during the binding process. Here, one would obtain a sample suspected of containing a wild type or mutant lipocalin protein and/or peptide, and contact the sample with an antibody against wild type or mutant lipocalin, and then detect and quantify the amount of immune complexes formed under the specific conditions.

In terms of antigen detection, the biological sample analyzed may be any sample that is suspected of containing a wild type or mutant lipocalin protein-specific antigen, such as a breast or prostate tissue section or specimen, a homogenized tissue extract, a lipocalin cell, separated and/or purified forms of any of the above wild type or mutant lipocalin protein-containing compositions, or even any biological fluid that comes into contact with the bone marrow cells and/or tissue, including blood and/or serum, although tissue samples or extracts are preferred. Hyperproliferative diseases that may be suspected of containing a wild type or mutant lipocalin protein-specific antigen include, but are not limited to, the collection of conditions classified as cancer therapy.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any lipocalin protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The lipocalin antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various forms of hyperproliferative diseases, such as cancer, including leukemia, for example. Here, a biological and/or clinical sample suspected of containing a wild type or mutant lipocalin protein, polypeptide, peptide and/or mutant is used. However, these embodiments also have applications to non-clinical samples, such as in the titering of antigen or antibody samples, for example in the selection of hybridomas.

In the clinical diagnosis and/or monitoring of patients with various forms of hyperproliferative disease, such as cancer, for example, leukemia, the detection of lipocalin mutant, and/or an alteration in the levels of lipocalin, in comparison to the levels in a corresponding biological sample from a normal subject is indicative of a patient with hyperproliferative disease, such as cancer, including leukemia. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive.

E. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the anti-lipocalin antibodies of the invention are immobilized onto a selected surface exhibiting protein affinity, such as a well in a polystyrene microtiter plate. Then, a test composition suspected of containing the wild type and/or mutant lipocalin protein antigen, such as a clinical sample, is added to the wells. After binding and/or washing to remove non-specifically bound immune complexes, the bound wild type and/or mutant lipocalin protein antigen may be detected. Detection is generally achieved by the addition of another anti lipocalin antibody that is linked to a detectable label. This type of ELISA is a simple “sandwich ELISA”. Detection may also be achieved by the addition of a second anti-lipocalin antibody, followed by the addition of a third antibody that has binding affinity for the second antibody, with the third antibody being linked to a detectable label.

In another exemplary ELISA, the samples suspected of containing the wild type and/or mutant lipocalin protein antigen are immobilized onto the well surface and/or then contacted with the anti-lipocalin antibodies of the invention. After binding and/or washing to remove non-specifically bound immune complexes, the bound anti-lipocalin antibodies are detected. Where the initial anti-lipocalin antibodies are linked to a detectable label, the immune complexes may be detected directly. Again, the immune complexes may be detected using a second antibody that has binding affinity for the first anti lipocalin antibody, with the second antibody being linked to a detectable label.

Another ELISA in which the wild type and/or mutant lipocalin proteins, polypeptides and/or peptides are immobilized, involves the use of antibody competition in the detection. In this ELISA, labeled antibodies against wild type or mutant lipocalin protein are added to the wells, allowed to bind, and/or detected by means of their label. The amount of wild type or mutant lipocalin protein antigen in an unknown sample is then determined by mixing the sample with the labeled antibodies against wild type and/or mutant lipocalin before and/or during incubation with coated wells. The presence of wild type and/or mutant lipocalin protein in the sample acts to reduce the amount of antibody against wild type or mutant protein available for binding to the well and thus reduces the ultimate signal. This is also appropriate for detecting antibodies against wild type or mutant lipocalin protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

F. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in 70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

G. Immunoelectron Microscopy

The antibodies of the present invention may also be used in conjunction with electron microscopy to identify intracellular tissue components. Briefly, an electron-dense label is conjugated directly or indirectly to the anti-lipocalin antibody. Examples of electron-dense labels according to the invention are ferritin and gold. The electron-dense label absorbs electrons and can be visualized by the electron microscope.

H. Immunodetection Kits

In still further embodiments, the present invention concerns immunodetection kits for use with the immunodetection methods described above. As the lipocalin antibodies are generally used to detect wild type and/or mutant lipocalin proteins, polypeptides and/or peptides, the antibodies will preferably be included in the kit. However, kits including both such components may be provided. The immunodetection kits will thus comprise, in suitable container means, a first antibody that binds to a wild type and/or mutant lipocalin protein, polypeptide and/or peptide, and/or optionally, an immunodetection reagent and/or further optionally, a wild type and/or mutant lipocalin protein, polypeptide and/or peptide.

In preferred embodiments, monoclonal antibodies will be used. In certain embodiments, the first antibody that binds to the wild type and/or mutant lipocalin protein, polypeptide and/or peptide may be pre-bound to a solid support, such as a column matrix and/or well of a microtitre plate.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present invention.

The kits may further comprise a suitably aliquoted composition of the wild type and/or mutant lipocalin protein, polypeptide and/or polypeptide, whether labeled and/or unlabeled, as may be used to prepare a standard curve for a detection assay. The kits may contain antibody-label conjugates either in fully conjugated form, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. Where wild type and/or mutant lipocalin protein, polypeptide and/or peptide, and/or a second and/or third binding ligand and/or additional component is provided, the kit will also generally contain a second, third and/or other additional container into which this ligand and/or component may be placed. The kits of the present invention will also typically include a means for containing the antibody, antigen, and/or any other reagent containers in close confinement for commercial sale. Such containers may include injection and/or blow-molded plastic containers into which the desired vials are retained.

### IX. Kits of the Invention

In particular embodiments of the invention, there is a kit housed in a suitable container. The kit may be suitable for cancer therapy for an individual or for cancer diagnosis, or both. In particular embodiments, the kit comprises in a suitable container an agent that targets lipocalin, such as targets its secretion, its expression, its activity, its secreted form, or a combination thereof. The agent may be an antibody, a small molecule, a polynucleotide, a polypeptide, a peptide, or a mixture thereof. The agent may be provided in the kit in a suitable form, such as sterile, lyophilized, or both, for example.

The kit may further comprise one or more apparatuses for delivery of the agent to an individual in need thereof. The apparatuses may include a syringe, eye dropper, needle, biopsy tool, scoopula, catheter, and so forth.

In embodiments wherein the kit is employed for a diagnostic purpose, the kit may further provide one or more detection compositions or apparatuses for identifying a secreted form of lipocalin. Such an embodiment may employ a detectable label, such as for an antibody, for example, and the label may be fluorescent, chemiluminescent, calorimetric, and so forth.

### X. Nucleic Acid-Based Expression Systems

The present invention concerns delivering a lipocalin-inhibiting substance, which may be referred to as an agent, to an individual in need thereof, such as an individual with cancer. In particular embodiments, the lipocalin-inhibiting substance affects expression of lipocalin, such as with anti-sense RNA, siRNA, or both, for example. The following description concerns exemplary reagents and methods for nucleic acid delivery, although in a specific embodiment a nucleic acid is delivered by lentivirus.

1. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaiyotic cell, and promoters or enhancers not “naturally occurring,” i.e., containing different elements of different transcriptional regulatory regions, and/or mutations that alter expression. For example, promoters that are most commonly used in recombinant DNA construction include the β lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In specific embodiments, the promoter is suitable for use in a cancer cell, such as a leukemia cell, a breast cancer cell, or a prostate cancer cell, for example.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al, 1998), murine epididymal retinoic acid-binding gene (Lareyre et al., 1999), human CD4 (Zhao-Emonet et al, 1998), mouse alpha2 (XI) collagen (Tsumaki, et al., 1998), DIA dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEMTM 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with beta galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

j. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention may comprise a viral vector that encode one or more compositions or other components such as, for example, an immunomodulator or adjuvant. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the compositions of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have useful as delivery vectors due to their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding a composition of interest) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mam et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985), and any combination of such methods. Through the application of techniques such as these, organelle(s), cell(s), tissue(s) or organism(s) may be stably or transiently transformed.

a. Ex Vivo Transformation

Methods for transfecting vascular cells and tissues removed from an organism in an ex vivo setting are known to those of skill in the art. For example, canine endothelial cells have been genetically altered by retroviral gene transfer in vitro and transplanted into a canine (Wilson et al., 1989). In another example, yucatan minipig endothelial cells were transfected by retrovirus in vitro and transplated into an artery using a double-ballonw catheter (Nabel et al., 1989). Thus, it is contemplated that cells or tissues may be removed and transfected ex vivo using the nucleic acids of the present invention. In particular aspects, the transplanted cells or tissues may be placed into an organism. In preferred facets, a nucleic acid is expressed in the transplated cells or tissues.

b. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of composition used may vary upon the nature of the antigen as well as the organelle, cell, tissue or organism used

c. Electroporation

In certain embodiments of the present invention, a nucleic acid is introduced into an organelle, a cell, a tissue or an organism via electroporation. Electroporation involves the exposure of a suspension of cells and DNA to a high voltage electric discharge. In some variants of this method, certain cell wall degrading enzymes, such as pectin degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre B lymphocytes have been transfected with human kappa immunoglobulin genes (Potter et al., 1984), and rat hepatocytes have been transfected with the chloramphenicol acetyltransferase gene (Tur Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon derived protoplasts is described by Dhir and Widholm in International Patent Application No. WO 9217598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

d. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV 1, BHK, NIH3T3 and HeLa cells were transfected with a neomycin marker gene (Chen and Okayama, 1987), and rat hepatocytes were transfected with a variety of marker genes (Rippe et al., 1990).

e. DEAE Dextran

In another embodiment, a nucleic acid is delivered into a cell using DEAE dextran followed by polyethylene glycol. In this manner, reporter plasmids were introduced into mouse myeloma and erythroleukemia cells (Gopal, 1985).

f. Sonication Loading

Additional embodiments of the present invention include the introduction of a nucleic acid by direct sonic loading. LTK fibroblasts have been transfected with the thymidine kinase gene by sonication loading (Fechheimer et al., 1987).

g. Liposome Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome mediated nucleic acid delivery and expression of foreign DNA in vitro has been very successful (Nicolau and Sene, 1982; Fraley et al., 1979; Nicolau et al., 1987). The feasibility of liposome mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome encapsulated DNA (Kaneda et al., 1989). In other embodiments, a liposome may be complexed or employed in conjunction with nuclear non histone chromosomal proteins (HMG 1) (Kato et al., 1991). In yet further embodiments, a liposome may be complexed or employed in conjunction with both HVJ and HMG 1. In other embodiments, a delivery vehicle may comprise a ligand and a liposome.

h. Receptor Mediated Transfection

Still further, a nucleic acid may be delivered to a target cell via receptor mediated delivery vehicles. These take advantage of the selective uptake of macromolecules by receptor mediated endocytosis that will be occurring in a target cell. In view of the cell type specific distribution of various receptors, this delivery method adds another degree of specificity to the present invention.

Certain receptor mediated gene targeting vehicles comprise a cell receptor specific ligand and a nucleic acid binding agent. Others comprise a cell receptor specific ligand to which the nucleic acid to be delivered has been operatively attached. Several ligands have been used for receptor mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell specific binding. For example, lactosyl ceramide, a galactose terminal asialganglioside, have been incorporated into liposomes and observed an increase in the uptake of the insulin gene by hepatocytes (Nicolau et al., 1987). It is contemplated that the tissue specific transforming constructs of the present invention can be specifically delivered into a target cell in a similar manner.

i. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95/06128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occuning cells which do not contain a recombinantly introduced nucleic acid.

In certain embodiments, it is contemplated that RNAs or proteinaceous sequences may be co expressed with other selected RNAs or proteinaceous sequences in the same host cell. Co expression may be achieved by co transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed with a composition of the invention. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, anthers, ascite tissue, cobs, ears, flowers, husks, kernels, leaves, meristematic cells, pollen, root tips, roots, silk, stalks, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, webpage http://phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F, lambda, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as beta mercaptoethanol or DTT (dithiothreitol), and refolded into a more desirable conformation, as would be known to one of ordinary skill in the art.

### XI. Proteins, Polypeptides, and Peptides

The present invention also provides purified, and in preferred embodiments, substantially purified, proteins, polypeptides, or peptides. The term “purified proteins, polypeptides, or peptides” as used herein, is intended to refer to an proteinaceous composition, isolatable from mammalian cells or recombinant host cells, wherein the at least one protein, polypeptide, or peptide is purified to any degree relative to its naturally obtainable state, i.e., relative to its purity within a cellular extract. A purified protein, polypeptide, or peptide therefore also refers to a wild type or mutant protein, polypeptide, or peptide free from the environment in which it naturally occurs.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases. The coding regions for these known genes may be amplified and/or expressed using the techniques disclosed herein or by any technique that would be know to those of ordinary skill in the art. Additionally, peptide sequences may be sythesized by methods known to those of ordinary skill in the art, such as peptide synthesis using automated peptide synthesis machines, such as those available from Applied Biosystems (Foster City, Calif.).

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of a specific fusion protein using a specific binding partner. Such purification methods are routine in the art. As the present invention provides DNA sequences for the specific proteins, any fusion protein purification method can now be practiced. This is exemplified by the generation of an specific protein glutathione S transferase fusion protein, expression in E. coli, and isolation to homogeneity using affinity chromatography on glutathione agarose or the generation of a polyhistidine tag on the N or C terminus of the protein, and subsequent purification using Ni affinity chromatography. However, given many DNA and proteins are known, or may be identified and amplified using the methods described herein, any purification method can now be employed.

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Methods exhibiting a lower degree of relative purification may have advantages in total recovery of protein product, or in maintaining the activity of an expressed protein. Inactive products also have utility in certain embodiments, such as, e.g., in determining antigenicity via antibody generation.

### XII. Examples

The following examples are offered by way of example, and are not intended to limit the scope of the invention in any manner.

### Example 1

### Mouse Hematopoietic Cells Expressing BCR-ABL Persistently Express 24P3 Transcripts

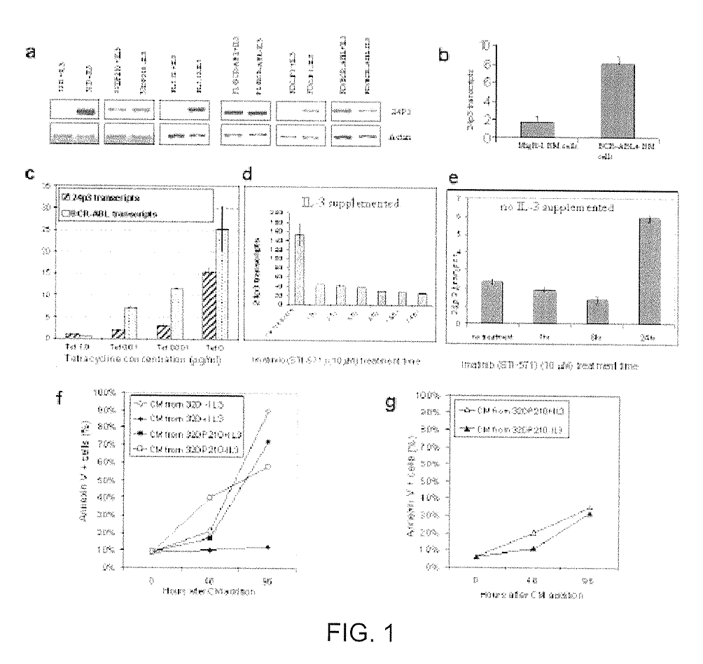

Using RT-PCR, 24p3 RNA expression was found in several IL-3 dependent mouse hematopoietic cell lines transformed by BCR-ABL (FIG. 1a). Importantly, 24p3 expression was independent of IL-3 (FIG. 1a). Transformation of these cells with BCRABL abrogates the need for IL-3 for cell proliferation and survival (Daley et al., 1987).

BCR-ABL negative counterparts of these cell lines require IL-3 for proliferation and survival, and produce only trace levels of 24p3 unless starved of IL-3 (FIG. 1a). In contrast, BCR-ABL+ mouse hematopoietic cells constitutively produce transcripts of 24p3 independent of IL-3 (FIG. 1a). Similar results were obtained with BaF3-BCR-ABL cell system (not shown). Importantly, transduction of BCR-ABL into primary mouse marrow cells also greatly stimulated expression of 24p3 transcripts (FIG. 1b).

### Example 2

### 24P3 Expression of BCR-ABL+ Cells Requires the Tyrosine Kinase of BCR-ABL

The present inventors examined the requirements for 24p3 expression in BCR-ABL+ 32D cells. The present inventors used the tetracycline (Tet) induction system to determine whether a reduction in BCR-ABL levels would reduce the level of 24p3 transcripts. Cells maintained in a high dose of Tet to suppress BCR-ABL expression had reduced levels of 24p3 transcripts relative to no Tet. Increased expression of BCR-ABL as measured by RT-PCR correlated with increased expression of 24p3 transcripts (FIG. 1c). Treatment of cells with imatinib mesylate (IM), a potent inhibitor of the Bcr-Abl tyrosine kinase (Druker et al., 2002) in the presence of IL-3 to prevent apoptosis induction, greatly reduced 24p3 transcripts (FIG. 1d). In the absence of IL-3, the level of transcripts showed a modest decrease but after 8 hrs the level of 24p3 transcripts more than doubled (FIG. 1e), suggesting that the mechanism of the induction of 24p3 by Bcr-Abl is different from the mechanism of 24p3 induction utilized by IL-3 starvation of normal 32D cells (FIG. 1e). It is important to note that in the absence of IL-3 and in the presence of IM, these BCR-ABL+ cells immediately begin the process of stimulating 24p3 expression. Therefore, the reduction of 24p3 transcripts by IM at early times (e.g. 1 hr), which is seen in FIG. 1d, is not as vigorous when IL-3 is absent (FIG. 1e) due to what appears to be two distinct mechanisms of 24p3 induction. Nevertheless, these results indicate that the tyrosine kinase activity of the Bcr-Abl oncoprotein is required for persistent induction of 24p3 expression.

### Example 3

### BCR-ABL+ 32D Cells are Resistant to the Apoptotic Effects of Conditioned Medium from Cells Expressing 24P3

Since P210 BCR-ABL is known to activate Stat5, to increase expression of BCL-XL and BCL-2 proteins in a manner that is independent of the IL-3 receptor pathway, and inhibit the activation of Bad and that Bad and Bcl-XL antagonize each other effect on cell death (Salomoni et al., 2000), it was determined whether BCR-ABL expression in hematopoietic cells rendered them resistant to the apoptotic effects induced by 24p3.

Indeed as expected 32D cells were sensitive to apoptosis induction by conditioned medium (CM) from either IL-3 starved 32D cells or BCR-ABL+ 32D cells (FIG. 1f). In contrast, BCR-ABL+ 32D cells were resistant to the apoptotic effects of CM from either IL-3 starved 32D cells or BCR-ABL+ 32D cells (FIG. 1g; Table 1). CM from BCR-ABL+32D cells also induced apoptosis in normal mouse bone marrow cells maintained in primary culture and 32D cells, but again BCR-ABL+ 32D cells were resistant to this treatment (Table 1). Importantly, CM from COS1 cells transfected with the 24p3 cDNA also induced apoptosis in primary mouse marrow cells and 32D cells but not BCR-ABL+ 32D cells (Table 1). Vector transfected COS1 cells lacked this activity (not shown).

TABLE 1
Bcr-Abl + 32D and IL-3 deprived 32D cells secrete a factor that
induced apoptosis in diploid murine bone marrow (BM) cells
and 32D cells, but BCRABL + 32D cells were resistant to the
apoptotic effects

| | Annexin V positive target cells | | |
|---|---|---|---|
| Source of | Murine primary | | BCR-ABL + 32D |
| conditioned medium | BM cells | 32D cells | cells |
| Medium + IL-3 | 5.8% | 1.6% | N/D |
| Medium without IL-3 | 10% | 27% | 0.9% |
| CM from BCR- | 34% | 78% | 0.9% |
| ABL + 32D cells | | | |
| CM from IL-3 starved | 84% | 66% | 1.5% |
| 32D cells | | | |
| CM from 24p3 COS1 | 42% | 29% | 0.9% |
| cells | | | |

CM was collected from cells with density of 2 × 10e6/ml after 48 hr. CM was filtered through 0.45 micron filter and stored at 4 deg. C. Target cells were exposed to CM for 48 hr prior to flow cytometry analyses. Target cells were treated in wells at about 500,000 cells per ml. Recombinant IL-3 was added at 3 ng/ml to the bone marrow culture, only. COS1 cells were transiently transfected with 24p3 in a pcDNA3 plasmid for 48 hr. CM was collected as above.
N/D. = Not Done.

### Example 4

### BCR-ABL+ 32D Cells Secrete 24P3

Western blotting detected the 24p3 protein in BCR-ABL+ 32D cells (P210 form in a mouse myeloid lineage) (FIG. 2a,b) and in BCR-ABL+ FL5.12 cells (P210 form in a mouse lymphoid lineage) (not shown). In contrast, these cells when not expressing BCR-ABL had reduced levels of 24p3 protein unless deprived of IL-3 (FIG. 2c). As expected, the 24p3 protein was also secreted into the culture medium of cells expressing BCR-ABL (FIG. 2c).

### Example 5

### Expression of Anti-Sense 24P30R siRNAs Inhibits 24P3 Protein Expression

To achieve efficient and rapid gene transduction in hematopoietic cells, the present inventors adopted a modified lentivitus gene transfer method (Ling et al., 2003). BCR-ABL+ 32D cells were transduced with an anti-sense 24p3 sequence and enhanced green fluorescent protein (GFP) using infection with a bi-cistronic lentivirus (Ling et al., 2003). The bicistronic lentivirus yields a viral mRNA that produces both anti-sense 24p3 and GFP.

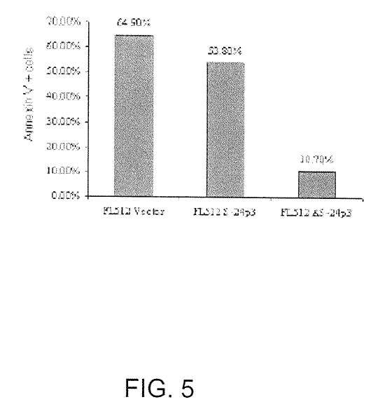

The EF1α promoter and the SIN modification (to eliminate the LTR promoter) was used to enhance expression in hematopoietic cells (Ling et al., 2003). Cell sorting by flow cytometry was used to enrich the cultured cell population (more than 90%) for antisense 24p3/GFP expression. Lentivirus infected cells expressed anti-sense 24p3 RNA, as determined by RT-PCR using a sense 24p3 primer and anti-sense GFP primer (not shown). Western blotting established that 24p3 protein levels were diminished relative to vector control BCR-ABL+ 32D cells in cells and in culture medium (FIG. 2a,b). In separate experiments, lentivirus infection was used to transduce sense 24p3. Sense 24p3 expression increased the amount of 24p3 in BCR-ABL+ 323Dcells (FIG. 2a) and in culture medium (not shown). Two 24p3 siRNAs were separately transduced into BCR-ABL+ 32D cells using a lentivirus siRNA/GFP vector (Wiznerowicz and Trono, 2003). Cell sorting produced cell populations that were more than 90% positive for GFP. Western blotting indicated that levels of 24p3 protein in these cells were reduced 75-80% (FIG. 2b). CM from 24p3 anti-sense and 24p3 siRNA expressing cultures had significantly depressed levels of 24p3 protein (FIG. 2c). As another indication that the anti-sense 24p3 produces low levels of 24p3, the inventors transduced anti-sense 24p3 into FL5.12 cells and determined whether IL-3 starvation would cause less apoptosis than vector transduced cells. Expression of anti-sense 24p3 in FL5.12 cells greatly reduced apoptosis induction (11%) compared to vector control (65%) when cells were maintained for 48 hr in the absence of IL-3 (FIG. 5).

The present inventors determined whether the presence of 24p3 protein in CM correlated with its known property of inducing apoptosis. CM from 32D cells deprived of IL-3 induced apoptosis in 32D cells and FL512 cells, which were maintained in presence of 3 ng/ml IL-3 during treatment to maintain viability and to prevent induction of 24p3 (FIG. 2d).

As expected, CM from 32D cells maintained in IL-3 did not induce apoptosis. CM from BCR-ABL+ 32D cells expressing the either the GFP gene (not shown) or sense 24p3 induced high levels of apoptosis in 32D target cells (FIG. 2d). In contrast, BCRABL+32D cells expressing either anti-sense 24p3 or siRNA for 24p3 had a greatly reduced level of apoptotic activity (FIG. 2d).

The present inventors performed co-culture experiments to determine whether BCR-ABL+ 32D cells would takeover the culture in a mixture of 32D cells and BCR-ABL+ 32D cells (FIG. 2e). In these experiments, the present inventors co-cultured 32D cells lacking GFP expression with an equal number of either 32D cells expressing GFP only or BCR-ABL+ 32D/GFP cells. Viable cells were determined by trypan blue staining. The number of viable 32D cells was unchanged when co-cultured with 32D cells expressing GFP. In contrast, the number of 32D cells showed a dramatic reduction when co-cultured with BCR-ABL+ 32D/GFP cells (FIG. 2e). It should be noted that the proliferation rate of BCR-ABL positive and negative 32D cells was essentially the same over the 13 day time period. Similar results were obtained with co-culture of cells separated by a barrier that prevented mixing of cells (not shown).

### Example 6

### 24P3 Antibody Inhibits Induction of Cell Death Induced by 24P3

To provide evidence that 24p3 was required for the induction of apoptosis, it was determined whether an affinity purified antibody to 24p3 would inhibit the apoptotic inducing activity of CM from BCR-ABL+ 32D cells. CM from BCR-ABL+ 32D cells, supplemented with IL-3 (2 ng/ml) to maintain viable cells, was added to BALB/c primary bone marrow cells. Marrow cells were tested for apoptosis after mixing with either 2 ug of protein from pre-immune rabbit serum or 1.5 ug of protein from affinity purified antibody to 24p3 compared to untreated controls (FIG. 2f). The mouse marrow target cells were assayed by trypan blue staining in triplicate after 48 hr. The affinity-purified 24p3 antibody partially blocked the apoptotic activity (FIG. 2f), indicating that 24p3 was involved in apoptosis induction.

### Example 7

### Reduction of 24P3 Expression by BCR-ABL+ 32D Cells Extended Survival and Restored Hematopoiesis in Marrow and Spleen of Leukemic Mice

Injection of NOD/scid mice pre-conditioned by sub-lethal radiation with BCR-ABL+ mouse 32D cells caused a lethal leukemia syndrome (FIG. 3a) involving marrow and spleen tissue. The survival time of these mice after iv inoculation of the BCR-ABL+ cells varies between 15-25 days, and is dependent on the level of BCR-ABL expression.

Pathology studies of spleen and marrow revealed that BCR-ABL+ 32D/GFP cells induced vigorous infiltration of GFP+ tumor cells into spleens and marrow. These leukemic tissues had only few regions of active normal hematopoiesis in NOD/scid mice that would normally produce erythrocytes, myeloid cells and megakaryocytes (FIG. 3d-g).

The present inventors examined the effects of reduced 24p3 expression in BCR-ABL+ 32D cells on the level of normal hematopoiesis in marrow and spleens of NOD/scid mice. A significant increase in the survival time was observed in mice injected with BCR-ABL+ 32D cells that expressed anti-sense 24p3 (FIG. 3a). Microscopic analysis revealed that leukemic spleen tissue and marrow from the anti-sense 24p3/GFP group of mice had a significantly larger amount of normal hematopoiesis (FIG. 3c,f) compared to that of GFP vector control (FIG. 3d,g). Importantly, these tissues from anti-sense mice resembled those injected with 32D cells lacking BCR-ABL expression. Cause of death of death of mice injected with BCR-ABL+ 32D cells expressing anti-sense 24p3 appeared to be due to extensive liver involvement. In contrast when 24p3 expression was maintained, marrow and spleen tissues were heavily infiltrated with leukemia cells and had very little normal hematopoiesis (FIG. 3d,g), suggesting that 24p3 secretion by leukemic cells is responsible for the observed reduction of normal hematopoiesis.

Because the leukemia cells are expressing GFP, the inventors measured the engraftment of leukemic cells in marrow and spleen of leukemic mice. Anti-sense expression in BCRABL+32D cells reduced invasion of spleen by more than 35-fold (FIG. 4a) in leukemic mice between 16 and 18 days after injection. Similarly, invasion of bone marrow was reduced almost 20-fold by anti-sense expression. Spleens and marrow fluid from these mice were assessed for levels of 24p3 by Western blotting. Bone marrow fluid from mice injected with anti-sense expressing BCR-ABL+ 32D cells contained essentially no 24p3 protein whereas GFP expressing cells contained relatively high levels of 24p3.

Mice treated with radiation and not injected with leukaemia cells also h a d no detectable amount of 24p3 (FIG. 4b). Another measure of normal hematopoiesis is the blood platelet levels. The present inventors measured the level of platelets in blood from these leukemic mice. Mice injected with anti-sense 24p3 expressing BCR-ABL+ 32D cells had greatly increased blood platelets levels compared to GFP vector cells or cells expressing sense 24p3 (FIG. 4c). Similarly leukemic C3H/HeJ mice injected with BCR-ABL+ 32D cells had severely depressed platelet levels but again injection of cells expressing anti-sense 24p3 dramatically increased the levels of platelets in peripheral blood (FIG. 4c). These findings support the microscopic pathological findings which indicate that mice injected with anti-sense 24p3 expressing BCR-ABL+ 32D cells have enhanced levels of normal hematopoiesis including megakaryocytes (FIG. 3b-g).

The present inventors also measured levels of normal hematopoietic cells in the marrow and spleen of NOD/scid mice during 16-18 days after injecting leukaemia cells, at which time mice were sick from leukemia. In these experiments, GFP-negative cells were analyzed by flow cytometry with cell surface markers for erythroid and myeloid lineages. The results showed a dramatic increase erythroid precursors in marrow and spleen of leukemic mice injected with BCR-ABL+ 32D cells expressing anti-sense 24p3 (FIG. 4,d-g). The level of erythroid cells was similar to that in irradiated control mice. There was a similar increase in myeloid lineage cells in marrow of anti-sense 24p3 mice, although not as great as erythroid lineage cells (FIG. 4e). Of interest, there was an increase of non-erythroid/myeloid lineage cells in leukemic mice injected with GFP transduced BCR-ABL+32D cells compared to the anti-sense 24p3 mice or untreated control mice (FIG. 4f,i). These results indicate that reduced secretion of 24p3 by the BCR-ABL+ 32D cells induces a disease that has little involvement of the marrow and spleen, which are sites typically marked by substantial invasion with leukemic cells.

Based on the cell death findings and co-culture experiments in vitro, these results suggest that BCR-ABL+ 32D cells must induce cell death of normal hematopoietic cells of the marrow and spleen in order to allow for efficient replacement of normal hematopoietic cells with leukemia cells in these tissues.

### Example 8

### Expression of Anti-Sense 24P3 Does not Affect the Level OF BCR-ABL Expression and Cell Properties

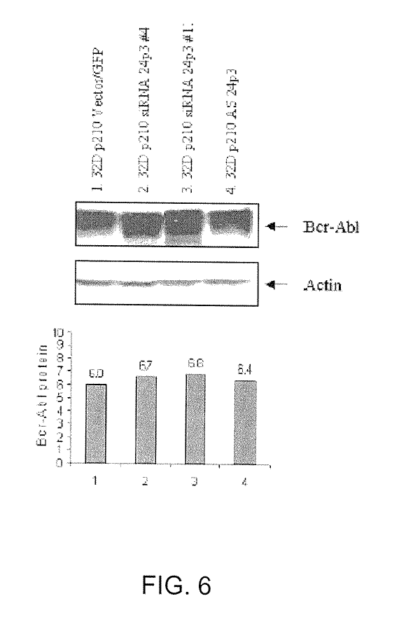

It is important to determine whether expression of anti-sense 24p3 in BCR-ABL+ 32D cells effects their oncogenic properties. The present inventors assessed the level of the Bcr-Abl oncoprotein in GFP vector control cells and cells transduced with anti-sense 24p3, and two siRNAs for 24p3 knockdown. Western blotting established that the level of BCRAbl protein expression was not affected after several months in cell culture (FIG. 6). In addition, the phosphotyrosine content of Bcr-Abl was also not affected by reduction of 24p3 (not shown). Similarly, 24p3 anti-sense expression did not affect the proliferation rate add cellular morphology (not shown). This is consistent with our findings that 24p3 anti-sense expression in leukemia cells still induced disease in mice with only a modest increase in survival (FIG. 3a). These mice show heavy invasion of leukemia cells in the liver, which appeared to be the cause of death. Importantly, experiments in the C3H/HeJ model showed that injection of BCR-ABL+ 32D cells expressing anti-sense 24p3 and 24p3 siRNA proliferated quite well in these mice, causing induction of solid tumors and ascites formation but like the NOD/scid model, these mice had little involvement of marrow and spleen (see Table 2).

TABLE 2
Reduction of 24p3 expression inhibits invasion of BCR-ABL+
32D cells in marrow and spleen of immunocompetent C3H/HeJ mice

| | Spleen | | | | | | | | |
|---|---|---|---|---|---|---|---|---|---|
| C3H mouse | wt | | Solid | GFP positive cells | | | | | |
| Groups | (mg) | Ascites | tumor | PB | BM | Spleen | Liver | Ascites | Tumor |
| 32Dp210 | 400 | N/A | N/A | N/A | N/A | N/A | N/A | N/A | N/A |
| Vector | | | | | | | | | |
| 32Dp210 | 250 | Yes | No | 75% | 80% | 55% | 60% | 100% | No |
| Vector | | | | | | | | | |
| 32Dp210 | 210 | No | Yes | 35% | 10% | 10% | 80% | No | 80% |
| Vector | | | | | | | | | |
| 32Dp210 | 960 | No | No | N/A | 90% | N/A | N/A | No | No |
| Vector | | | | | | | | | |
| 32Dp210 | 610 | No | No | 90% | 90% | 90% | 90% | No | No |
| Vector | | | | | | | | | |
| 32Dp210 AS | 110 | N/A | N/A | N/A | N/A | N/A | N/A | N/A | N/A |
| 32Dp210 AS | 260 | Yes | Yes | <1% | 5% | 1% | 10% | 100% | 90% |
| 32Dp210 AS | 170 | Yes | Yes | 10% | <1% | <1% | 5% | 80% | 60% |
| 32Dp210 AS | 160 | Yes | Yes | 5% | 2% | 2% | 40% | 80% | 100% |
| 32Dp210 AS | 130 | Yes | Yes | <1% | 5% | 2% | 55% | 100% | 90% |
| 32Dp210 siRNA | 160 | Yes | Yes | N/A | <1% | 5% | 20% | 90% | N/D |
| 32Dp210 siRNA | 110 | Yes | Yes | 50% | 5% | 25% | 50% | 80% | N/D |
| 32Dp210 siRNA | 40 | Yes | Yes | <1% | 5% | <1% | 40% | 100% | N/D |
| 32Dp210 siRNA | 90 | Yes | Yes | N/A | No | No | 10% | 90% | 40% |
| 32Dp210 siRNA | 130 | Yes | Yes | 20% | 5% | 20% | 25% | 100% | 70% |

Protocol and processing methods are similar to that described in the legends of FIG. 3 and FIG. 4 except the mice were not pre-conditioned by radiation. BCR- ABL+ 32D cells expressing either GFP or antisense 24p3 or siRNA #4 were used in this study, as in FIG. 2.
N/A: not analyzable.
N/D, not done.

### Example 8

### Homing of BCR-ABL+ 32D Cells to Various Tissues of Leukemic NOD/SCID Mice is not Affected by Anti-Sense 24P3 Expression

One question about 24p3 expression is whether it affects homing of BCR-ABL+ 32D cells in NOD/scid mice. To address this question, the present inventors searched for engraftment of BCRABL DNA in mice at 7 days after injection into mice. The present inventors used primers that detect the b3a2 junction of BCR-ABL. One round of PCR did not detect junction BCR-ABL sequences in various tissues. Nested PCR on DNA from various tissues detected junction sequences in all tissues examined but no differences were observed in BCRABL junction sequences of mice injected with BCR-ABL+ 32D cells expressing either GFP or anti-sense 24p3/GFP (Table 3).

TABLE 3
Homing of BCR-ABL + 32D cells in NOD/scid mice is not
affected by anti-sense 24p3 expression.

| | DNA PCR |
|---|---|
| AS PB | 0 |
| P210 PB | 0 |
| AS BM | 106 |
| P210 BM | 97 |
| AS Spleen | 81 |
| P210 Spleen | 77 |
| AS Liver | 46 |
| P210 Liver | 51 |

Seven days after i.v. injection of either 10e6 Vector/GFP BCR-ABL P210 32D cells or AS24p3 BCR-ABL P210 32D cells per mouse, mice were sacrificed to get peripheral blood (PB), bone marrow (BM), spleen and liver cells for genomic DNA extraction. DNA PCR was performed to measure the BCR-ABL junction in cellular DNA, and results were normalized for the amount of starting cellular DNA and analyzed (μg/ml). Nested DNA PCR was done two times on each sample. Average values of BCR-ABL DNA copies from two mice in each group were calculated and shown in the table. There is no statistical significant difference in the number of BCR-ABL DNA copies between the BCR-ABL+32D vector group and the BCR-ABL+ 32D anti-sense 24p3 group as determined by a paired t-test. (P=0.6450).

Anti-sense 24p3 and siRNA directed against 24p3 restores hematopoiesis in marrow and spleen of C3H/HeJ mice injected with BCR-ABL+ 32D cells BCR-ABL+ 32D cells have been shown to induce leukemia in the C3H/HeJ mouse strain without pre-conditioning regimens (Matulonis et al., 1995). Therefore, the present inventors wanted to determine whether the affects of anti-sense 24p3 expression in leukemia cells would produce the same effects as in the NOD/scid mouse. In these experiments, 10e6 BCR-ABL+32D cells were injected iv into 8-week old C3H/HeJ mice. Leukemia cells expressed either GFP or anti-sense 24p3 RNA or siRNA for 24p3. The GFP expressing leukemia cells induced a vigorous leukemia involving marrow, spleen, blood and liver (Table 2). Expression of anti-sense 24p3 in leukemia cells reduced the level of invasion in marrow and the spleen but not the liver. Importantly, these mice developed a vigorous ascites tumor growth of GFP+ cells, which was not observed in the GFP vector expressing leukemia cells. Similar results were observed in mice injected with BCRABL+32D cells expressing shRNA against 24p3 (Table 2). Thus, the results of studies in two different mouse strains indicate that reduction of 24p3 expression levels strongly reduced the invasion of marrow and spleen but not liver, and in the case of the C3H mouse strain, reduction of 24p3 expression also caused a vigorous growth of leukemia cells in the form of an ascites.

### Example 9

### 21 KDA form of NGAL is the Major form Secreted by BCR-ABL+ CML Cells

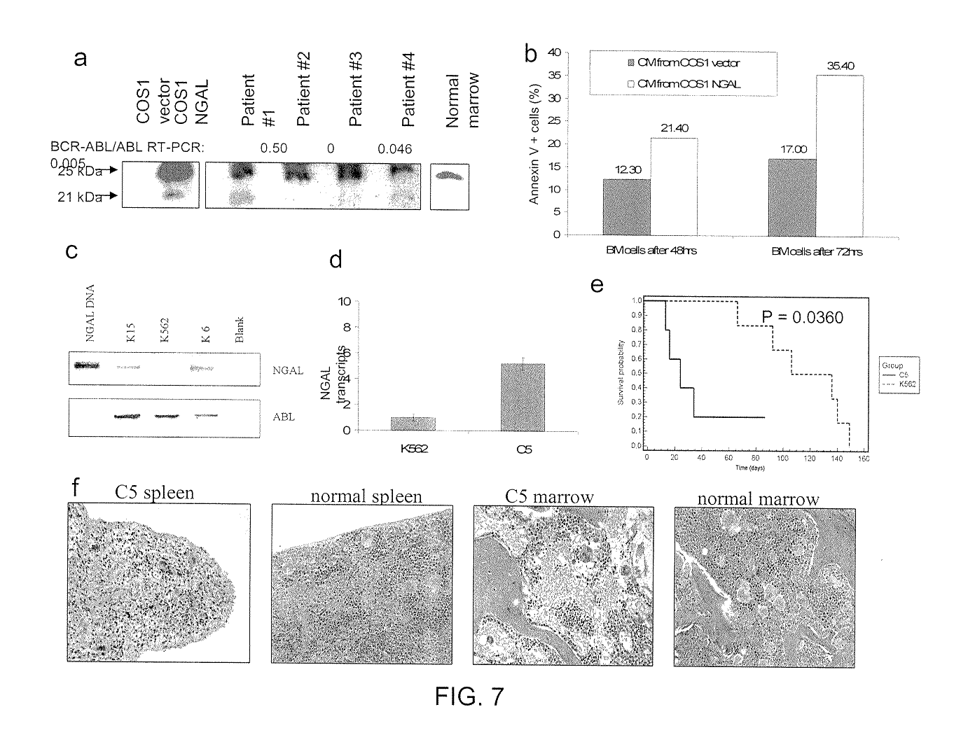

The present inventors examined NGAL production in CML cells. As with 24p3, NGAL secreted by marrow cells of CML patients had a smaller size (21 kDa) (FIG. 7a). This 21 kDa NGAL protein was secreted by marrow cells from CML patients having various levels of BCR-ABL but not from normal marrow or marrow cells from a CML patient with undetectable BCR-ABL. Mass spectrometry/protein sequence analysis of the 21 kDa form verified the presence of NGAL sequences in the 21 kDa NGAL gel band. CM from COS-1 cells transfected with NGAL cDNA had increased levels of apoptotic activity compared to vector transfected cells when assayed on primary mouse marrow cell targets (FIG. 7b). In support of our earlier study 4, soft agar selected clones of K562 cells, which induced aggressive leukemia and atrophy, produced relatively higher levels NGAL transcripts compared to un-cloned parental K562 cells (FIG. 7c). The high NGAL-producing clone of K562 cells (c5) induced early death in NOD/scid mice whereas uncloned parental K562 cells with a low-level of NGAL expression had a delayed onset disease and death (FIG. 7d,e). The c5 K562 clone caused suppression of hematopoiesis in marrow and spleen (which led to death of the mice) (FIG. 7f) whereas the low NGAL producing un-cloned parental K562 cells induced a disease with longer latency (FIG. 7e) and solid tumor formation, with reduced involvement of marrow and spleen (not shown). These results are in agreement with earlier findings of atrophy induced by the K6 clone of K562 cells 4.

These findings indicate that the Bcr-Abl oncoprotein in addition to its oncogenic effects also induces secretion of an apoptotic factor (24p3/NGAL) that causes suppression of normal hematopoiesis. However, in contrast to an earlier report (Devireddy et al., 2001), the present inventors were unable to induce apoptosis in suitable targets cells with purified 24p3/NGAL (GST-bacterial method; Goetz et al., 2002). Detailed structural analysis has shown that NGAL is an iron binding protein, and that the ligand for NGAL is a catecholate-type siderophore (Goetz et al., 2002). Whether the siderophore binding function is related to 24p3/NGAL's ability to induce apoptosis is not known. In specific embodiments, the apoptotic activity of 24p3/NGAL may require either post-translational modification in eukaryotic cells or formation of a complex with one or more factors. Nevertheless, suppression of normal hematopoiesis by a mechanism that does not interfere with the proliferation and survival of BCR-ABL+ cells would confer a significant cell growth and survival advantage for the leukemic cell clone in the normal marrow environment. Local suppression of normal hematopoiesis by 24p3/NGAL secreted by leukemia cells would allow the leukemic clone to more readily compete, survive, infiltrate and proliferate in normal marrow and the spleen tissue environment. These findings suggest that secretion of an apoptosis-inducing molecule like 24p3/NGAL by the BCR-ABL+ human leukemia cells in CML would be important for the establishment of the leukemia clone at early stages of the leukemia process. Thus, as little as one pluripotent stem cell that has acquired BCR-ABL expression by forming the Philadelphia chromosome could survive and compete in the normally active marrow environment because of its ability to secrete a cell death factor (and possibly other factors; Eaves et al., 1998; Olofsson and Olsson, 1980; Olofsson and Olsson, 1980; Skold et al., 1999) to which it is resistant. The ensuing cell death in the surrounding marrow cells would facilitate expansion of the leukemic clone in the normal marrow. It remains to be seen whether the suppression of normal hematopoiesis caused by BCR-ABL+ cells expressing 24p3/NGAL or a similar factor has additional consequences that benefit the survival of the leukemia clone, such as reduced immune responses in the marrow directed towards the Ph-positive pluripotent stem cell and the more differentiated leukemic stem cells. For progression of the leukemia to a more aggressive stage, the results indicate that NGAL expression by the BCR-ABL cells will select for cells with relatively high levels of BCR-ABL expression per cell, as cells with relatively low levels of oncoprotein would tend to succumb to the cell death effects of 24p3/NGAL.

### Example 10

### Significance of the Invention

These findings indicate that the Bcr-Abl oncoprotein in addition to its oncogenic effects also induces the secretion of a lipocalin 2 (24p3) that causes apoptosis of marrow cells and BCR-ABL negative mouse hematopoietic cells lines in cell culture (FIGS. 1 and 2 and Table 1). The induction of 24p3 expression by BCR-ABL requires the presence of the Bcr-Abl oncoprotein (FIG. 1c) and the tyrosine kinase activity of Bcr-Abl (FIG. 1d). Importantly, BCR-ABL+ cell lines are resistant to the apoptotic effects of 24p3 (FIG. 1, Table 1). The resistance of BCR-ABL+ cells to 24p3 effects is consistent with the known mechanism of apoptosis induction by 24p3, which involves activation of the proapoptotic factor Bad. Bcr-Abl is known to prevent activation of Bad (Salomoni et al., 2000) and to stimulate expression of Bcl-XL and Bcl-2 (Salomoni et al., 2000).

To determine the function of 24p3, the inventors developed assays to measure apoptosis induction in target cells (FIG. 2) using CM from BCR-ABL+ cells. The present inventors also developed known genetic approaches to reduce expression of 24p3 by use of anti-sense and siRNAs directed against 24p3 RNA sequences. Expression of anti-sense 24p3 and two different siRNA sequences for 24p3 strongly reduced 24p3 protein expression in BCR-ABL+ 32D cells (FIG. 2a,b) and also strongly reduced levels of 24p3 protein in the culture medium (FIG. 2c). CM from BCR-ABL+ 32D cells induced apoptosis in two different BCR-ABL-negative mouse hematopoietic cell lines (FIG. 2d). Reduction of 24p3 secretion by anti-sense/siRNA expression strongly reduced apoptotic activity of CM from BCR-ABL+ 32D cells (FIG. 1d). Co-culture of 32D cells with BCR-ABL+ 32D cells allowed BCR-ABL+ cells to overtake the culture, despite the similar proliferation rates of these cells in culture separately (FIG. 2e).

The studies demonstrated that an affinity-purified antibody against 24p3 inhibited the apoptotic activity of CM from BCR-ABL+ cells (FIG. 1f). Thus, these findings indicate that 24p3 functions as an apoptosis-inducing factor in agreement with the findings of Devireddy et al. (2001). However, the present inventors observed that 24p3 appears to exist in complexes, and thus in some embodiments of the present invention 24p3 acts alone or in combination with other factors.

The co-culture experiments and CM findings predict that BCR-ABL+ leukemia cells would over-grow spleen and marrow tissue sites by inducing cell death of normal hematopoietic cells, and thereby providing space for expansion of the invading leukemia cells. Our studies mouse models support this concept. In NOD/scid mice and C3H/HeJ mice, BCR-ABL+ 32D cells over-grow the marrow and spleen of leukemic mice. However, reduction of 24p3 expression by anti-sense or siRNA methods restores the level of hematopoiesis in marrow and spleen and elevates platelet levels in the blood (FIG. 3, FIG. 4). Of interest, marrow fluid from these mice injected with BCR-ABL+ 32D cells expressing GFP contained 24p3 but marrow fluid from mice injected with anti-sense or untreated lacked 24p3 (FIG. 4b). In addition, NOD/scid mice injected with anti-sense expressing BCR-ABL+32D cells had a small but significant increase in survival (FIG. 3a). This is surprising in view of the fact that the level of the Bcr-Abl oncoprotein is unaffected by the reduction of 24p3 expression (FIG. 6).

One unlikely but possible explanation for the restoration of hematopoiesis in mice injected with 24p3 anti-sense expressing BCR-ABL+ 32D cells is that these leukemia cells have lost the ability to proliferate in mouse tissues. This is not the case, as liver invasion readily takes place (Table 2), solid tumors are formed and ascites formation also occurs (Table 2). Also, cell culture studies did not detect changes in the proliferation rate, morphology and levels of the BCR-ABL oncoprotein in either antisense 24p3 cultures or siRNA expressing cultures compared to the GFP vector control (FIG. 6). Thus, these findings support the concept that the reduction of 24p3 expression only interferes with invasion and growth of leukemia cells in marrow and the spleen.

The mechanisms of the effects that are caused by reduction of 24p3 in mice require further studies. Changes in homing of leukemia cells with reduced 24p3 expression does not appear to explain our results. Thus, our studies on homing 7 days after injection suggest that the leukemia cells home to many tissues and more importantly, the reduction of 24p3 did not affect the early engraftment of leukemia cells in various tissue sites (Table 3). In addition our preliminary studies suggest that normal marrow cells are undergoing apoptosis in leukemic tissues from mice where 24p3 expression is maintained since spleens of mice injected with BCR-ABL+ 32D cells had an increased level of apoptosis as measured by TUNEL staining (not shown).

Technical difficulties make it difficult to monitor death of hematopoietic cells in spleen/marrow tissues extracted from sick mice due to rapid clearing of dead cells and because of the rapid over-growth of BCR-ABL+ 32D cells that occurs at these sites (FIG. 3). However, the CM studies and co-culture studies suggest that the mechanism for efficient invasion and suppression of normal hematopoiesis in the marrow and spleen of leukemic mice expressing the full amount of 24p3 is due to induction of apoptosis in normal hematopoietic cells by the secreted 24p3 protein (FIGS. 2,3 and 4).

These findings have important implications at least for leukemia, such as for chronic myeloid leukemia (CML). This blood cancer is believed to originate in a pluripotent stem cell by fusion of parts of the BCR and ABL genes through the formation of the Philadelphia chromosome. Suppression of normal hematopoiesis by a mechanism that does not interfere with the proliferation and survival of BCR-ABL+ cells would confer a significant cell growth and survival advantage for the leukemic clone in the competing normal marrow environment. Local suppression of normal hematopoiesis by apoptosis induction caused by 24p3 secreted by the leukemia cells would allow the leukemic clone to more readily survive, expand and invade in normal marrow and the spleen.

These findings indicate that secretion of an apoptosis-inducing molecule like 24p3 by the BCR-ABL+ human leukemia cells in CML is important for the establishment of the leukemia clone at early stages of the leukemia process, in specific aspects of the invention. Thus, as little as one pluripotent stem cell that has acquired BCR-ABL expression by forming the Philadelphia chromosome could survive and compete in the normally active marrow environment because of its ability to secrete a cell death factor and possibly other factors (Eaves et al., 1998; Olofsson and Olsson, 1980a; Olofsson and Olsson, 1980a; Skold et al., 1999) to which it is resistant.

In this regard, CML cells were shown to overproduce and secrete elastase, resulting in the reduction of growth factors such as G-CSF, thereby giving advantage to Ph+hematopoiesis (EL-Ouriaghli et al., 2003). Other negative factors are secreted by CML cells including transforming growth factor-β (TGF-β), tumor necrosis factor-α (TNF-α), macrophage inflammatory protein-1 (MIP1α), and monocyte chemoattractantprotein-1 (MCP-1), and leukemia-associated inhibitor (LAI) (Eaves et al., 1998; Olofsson and Olsson, 1980a; Olofsson and Olsson, 1980a; Skold et al., 1999). LAI inhibits normal but not leukemia granulopoiesis (Olofsson and Olsson, 1980a; Olofsson and Olsson, 1980a; Skold et al., 1999). LAI was identified as neutrophil pro-proteinase 3, a member of the neutrophil serine protease family (Skold et al., 1999). None of these factors were identified as apoptosis-inducing factors, and all were based on studies conducted in cell culture assays.

In a previous study of NOD/scid mice injected with clones of a chronic myeloid leukemia (CML) cell line (K562 cells), the present inventors observed a leukemia syndrome involving not only an extramedullary leukemia but also a severe reduction of normal mouse hematopoiesis (termed atrophy) (Lin et al., 2001). Some of these mice died of a wasting syndrome (loss of weight) that involved suppression of hematopoiesis without extensive tumor cell invasion of the spleen and marrow as observed in a typical leukemia. The findings indicate these K562 cell clones have enhanced expression of NGAL (neutrophil gelatinase associated lipocalin) (Hui Lin, Tong Sun, and R. Arlinghaus, unpublished), which is the human counterpart of 24p3 (Kjeldsen et al., 1993; Yousefi and Simon 2002). Of interest NGAL is present in granules of neutrophils, as is LAI (Kjeldsen et al., 2000; Skold et al., 1999). One may determine whether NGAL is responsible for the suppression of hematopoiesis in spleen and marrow by clones of K562 cells that the present inventors observed in these previous experiments (Lin et al., 2001).

### Example 11

### Therapeutic Antibodies of the Invention

In particular aspects of the invention, an antibody to the cell death factor that blocks its cell killing effects is utilized. In particular, this is a monoclonal antibody. In specific embodiments, the antibodies are humanized antibodies, herein defined as comprising a part of a mouse antibody gene responsible for recognizing a specific antigen, such as the lipocalin of the invention, with other parts from a human antibody gene. Thus, the “humanized” monoclonal antibody is enough like a normal human antibody to avoid being destroyed by the patient's own immune system. For example, using recombinant DNA technology the present inventors prepare the humanized antibody by inserting the coding sequences for the protein sequences that bind to and inactivate the cell death factor into human antibody heavy chain sequence.

In specific embodiments, the present inventors will purify 24p3 and NGAL, and the purified proteins are injected into mice to produce monoclonal antibodies. A commercial source may be employed to produce monoclonal antibodies. The various antibodies are tested for their ability to block the apoptotic activity of both 24p3 (mouse) and NGAL (human). The mouse monoclonal antibody is purified and injected into mice having CML to characterize the ability to treat the leukemia. The antibody to NGAL is similarly studied. Blocking antibodies for NGAL are tested for toxic effects in normal mice before undertaking the steps to produce a recombinant antibody composed of human IgG chains (so-called heavy and light chains) contains the sequences that bind to and inactivate the apoptotic activity of NGAL.

### Example 12

### Co-Culture Studies

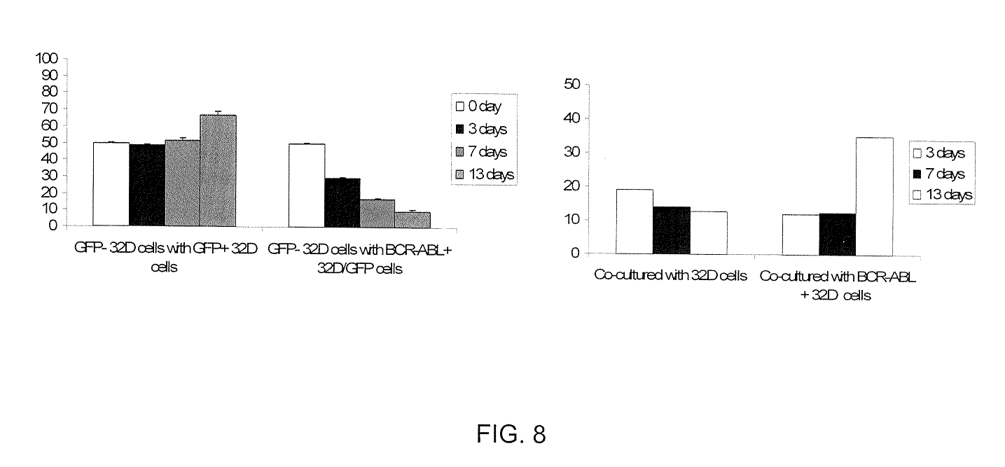

FIG. 8 shows co-culture studies of the present invention. In FIG. 8a, there is o-culture of BCR-ABL+ 32D cells with 32D cells without barrier decreases the number of 32D cells due to induction of apoptosis. GFP negative 32D cells (GFP-32D cells) were co-cultured with either 32D GFP+ cells or BCR-ABL+ 32D GFP cells in a 1:1 ratio in presence of 3 ng/ml recombinant IL-3. The cell cultures were diluted 2-fold with fresh culture medium with IL-3 every 2 days to maintain vigorous growth. The amount of viable GFP negative 32D cells were analyzed on day 0, 3, 7 and 13 by flow cytometry to determine those cells that were negative for GFP and Annexin V staining. b. Co-culture of BCR-ABL+ 32D cells with 32D cells in a culture dish with a barrier induces apoptosis of 32D cells. GFP-negative 32D cells were co-cultured with 32D GFP+ cells or BCR-ABL+ 32D GFP cells in a culture dish with a barrier to prevent cell mixing. All cells were grown in the culture medium with 3 ng/ml and diluted 2 fold every 2 days. Annexin V staining of GFP-negative 32D cells was determined by flow cytometry on days 3, 7 and 13.

### Example 13

### 24P3/NGAL Secretion in Breast Cancer

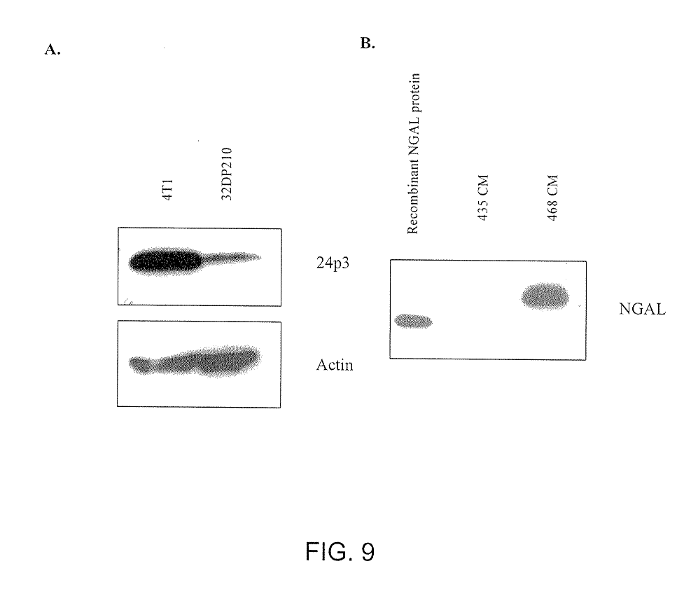

4p31NGAL is expressed and secreted by some breast cancer cell lines. In FIG. 9A, the mouse breast cancer cell line 4T1 and BCR-ABL(+) 32D(32DP210) cells (used as a positive control) were lysed and analyzed by Western blotting for 24p3. Actin was used as a loading control. In FIG. 9B, there is western blotting analysis of NGAL expression in conditioned medium (CM) from human breast cancer cell lines 435 and 468. After growing cells to confluency, standard medium was changed to serum-free medium for another 50 hours of cell culture. Equal volumes of CM was collected and concentrated in a Microcon YM-10 (Millipore) and subjected to Western blotting for NGAL. Recombinant NGAL was used as a positive control. The NGAL produced by the breast cancer cell line migrated at 24 kDa (the size of endogenous, unmodified NGAL). The 468 cells are known to invade bone in animal studies. The present inventors have examined several other breast cancer cell lines and found that 5 of 11 cell lines express NGAL. In particular embodiments, breast cancer cell lines that comprise activated EGFR secrete lipocalin.

### Example 14

### NGAL Expression in Prostate Cancer

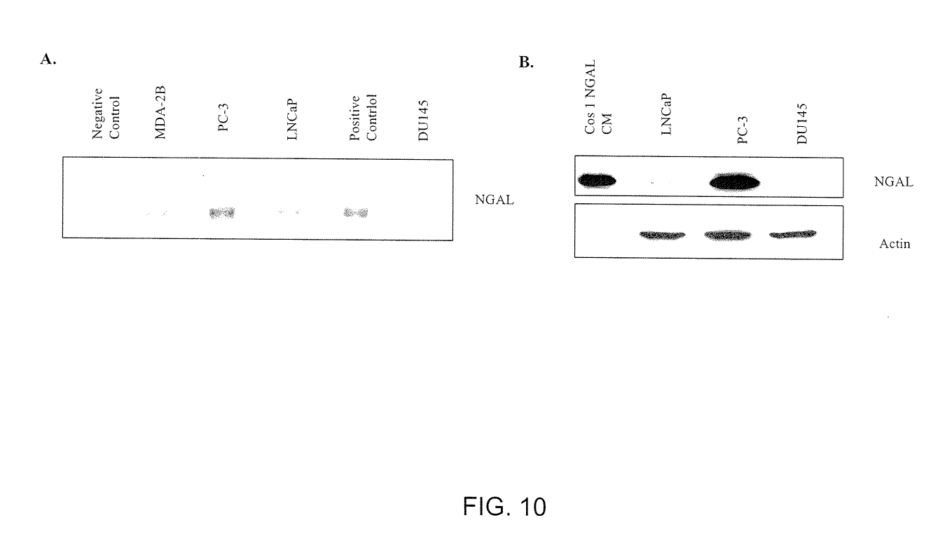

In particular embodiments, there is NGAL expression in human prostate cancer cell lines. In FIG. 10A, there are RT-PCR analyses of NGAL transcripts in different kinds of prostate cancer cells. RNA was extracted and same amount of RNA was used in the reverse transcriptase reaction to make cDNA. RT-PCR was performed by using same amount of cDNA. In FIG. 10B, there are western blotting analyses of NGAL expression in prostate cancer cells. Cell lysates from three different human prostate cell lines were examined by Western blotting with anti-NGAL. Conditioned Medium (CM) from NGAL transfected Cosl cells was used as a positive control for the 24 kDa form of NGAL. Actin was used as a loading control for the cell lysate samples. These results indicate that PC3 prostate cancer cells, known to invade bone in an animal model, expressed high levels of NGAL. In contrast, LNCap and DU145 express very little or no NGAL protein. These latter cells do not invade bone.

### Example 15

### Exemplary Methods and Materials

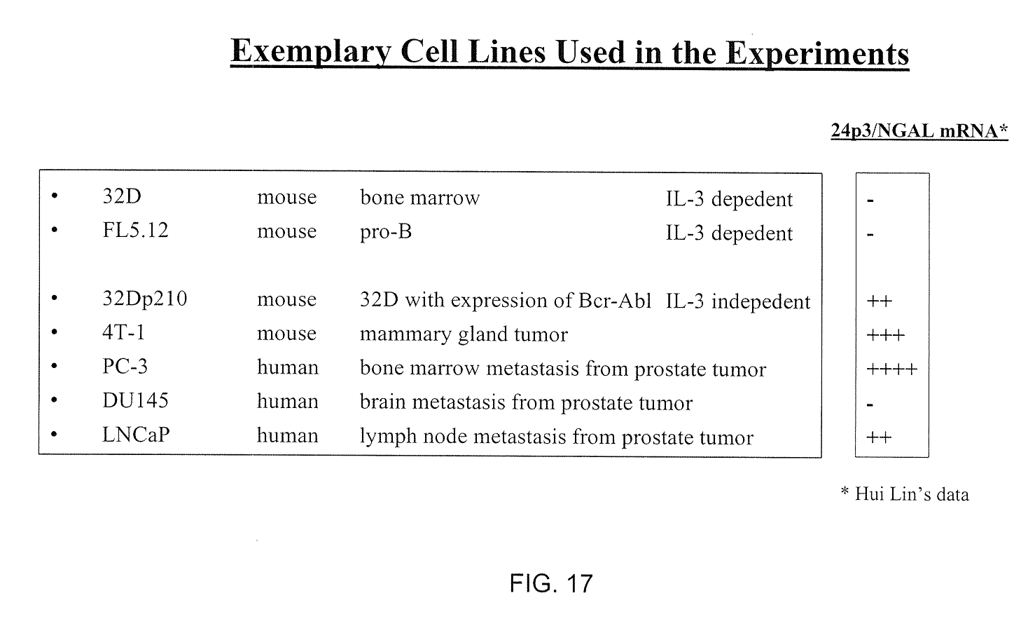

Cell lines, plasmid, lentivirus vectors. FIG. 17 shows characteristics of the exemplary cell lines employed herein.

The clone 3 line of 32D cells expressing P210 BCR-ABL (b3/a2) and other mouse hematopoietic cell lines (FL5.12, FDC-P1, BaF3) were maintained as described (McCubrey et al., 1993). 24p3 plasmid was provided by Michael Green (Univ. of Mass.) Both sense and anti-sense 24p3 were inserted into a lenitivirus transduction plasmid, which contains a bi-cistronic coding structure using an internal ribosomsal entry site (IRES) followed by the green fluorescent protein (GFP) gene (Ling et al., 2003). Transcription is driven by an EF-1α promoter (Ling et al., 1993). Lentiviruses encoding 24p3 siRNA were constructed as described (Ling et al., 2003; Wiznerowicz and Trono, 2003). Briefly, the siRNA is formulated as a small hairpin (sh) RNA driven by H1 promoter; the construct has EF-1α promoter to drive the GFP. The sequences of these constructs are: #4, GGCAGCTTTACGATGTACA-TTCAAGAGA-TGTACATCGTAAAGCTGCC-TTTTTTCCAAT (sense-loop-anti-sense-termination region) (SEQ ID NO:1); #11-CATTTGTTCCAAGCTCCAG-TTCAAGAGACTGGAGCTTGGAACAAATG-TTTTTTCCAAT (SEQ ID NO:2).

PCR and Western blotting. Total RNA was extracted and cDNA was prepared as described (Guo et al., 2002). 24p3 PCR amplimers were detected by RT-PCR using two oligonucleotide primers: Forward primer: AGCCAGACTTCCGGAGCGATC (SEQ ID NO:3); reverse primer: ACTTGGCAAAGCGGGTGAAACG (SEQ ID NO:4). For real-time RT-PCR: Probe: 24p3 p144 (DFAM)CCT GGC AGG CAA TGC GGT CC(DTAM) (SEQ ID NO:5); forward primer: 24p3 FW123 GGG CAG GTG GTA CGT TGT G (SEQ ID NO:6); reverse primer:24p3 RV190 CGT AAA GCT GCC TTC TGT TTT TTT (SEQ ID NO:7). Western blotting was performed as described (Xie et al, 2002). Conditioned medium was prepared as described (Devireddy et al, 2001).

Apoptosis assays were performed by flow cytometry to detect and quantitated by Annexin V reactivity. Mouse leukemia models. The NOD/scid model was used as described (Lin et al., 2001). 10e6 BCR-ABL+ 32D cells were injected iv, either vector transduced/GFP) or anti-sense 24p3/GFP. The C3H/HeJ mouse model was used as described (Matulonis et al, 1995); 1.0×10e6 cells were injected iv. Tissues were analyzed in our veterinary core facility. TUNEL staining was performed in the Division of Pathology core facility.

### Example 16

### Neutrophil Gelatinase-Associated Lipocalin 2 (NGAL) is Highly Associated with HER2+/ER− Breast Cancers and is a Downstream Effector of the PI3-K/AKT Pathway

As mentioned elsewhere herein, NGAL is a lipocalin 2 involved in inducing apoptosis in normal hematopoietic cells. The mouse form of NGAL (24p3) is required for marrow and spleen invasion in mouse leukemia models. The present inventors found that NGAL is over-expressed in tumor cells from human breast cancer patients. Importantly, over expression of NGAL correlates with patients that have a poor prognosis. In microarray studies, 21 of 133 breast cancer patients expressed very high levels (6-10 fold) of NGAL mRNA. Higher expression of NGAL correlated with increasing tumor size (P<0.013). The correlation with ER negativity and NGAL expression was also significant (P<0.001). Similarly, there was a strong positive correlation with HER2/neu expression (P<0.003). In addition, HER2+ER− patients also had significant levels of HER2/neu (P<0.01). Bone marrow grading for aggressiveness indicated that breast cancer tumors with median grade 3 had higher NGAL expression than grades less than 3 (P<0.003). The level of NGAL protein expression in cells and conditioned medium (CM) was much higher in the SKBr3 human breast cancer cell line (HER2/neu+, ER−) than MCF-7 cells (HER2/neu-, ER+). CM from SKBr3 cells induced apoptosis in mouse marrow cells. The Herceptin HER2 antibody and PI-3 kinase inhibitor LY294002 inhibited NGAL expression in the SKBr3 cell line. Activated Akt was also needed for optimum expression of NGAL. NGAL transcriptional sequences contain a consensus NF-kB binding site and since Akt is known to activate NF-kB, the inventors predict that NGAL expression is mediated through NF-kB. In that regard, a proteosome inhibitor (BAY11-7082), which is known to prevent IkB degradation, strongly inhibited expression of NGAL. Thus, in specific embodiments of the invention Her2/neu mediates NGAL expression in breast cancer cells, and the apoptotic induction activity of NGAL plays a role in bone marrow metastasis.

The following description provides further details to the discussion above in this Example. The previous experimental findings by the inventors indicated that the mouse lipocalin 2, 24p3, plays a major role in marrow invasion of leukemic mice by suppressing normal hematopoiesis. Blocking expression of 24p3 strongly inhibits invasion of marrow and spleen by BCR-ABL+ myeloid cells. In vitro studies with conditioned medium (CM) from leukemia cells indicate that 24p3 induces apoptosis in normal hematopoietic cells but not leukemia cells. Similarly, the CML cell clones that express high levels of NGAL strongly suppress hematopoiesis in marrow and spleen tissues, such as by a mechanism involving apoptosis induction, for example. Therefore, the present inventors examined human breast cancer cell tumor specimens for the expression of NGAL transcripts using microarray analysis and determined the mechanism of induction of NGAL in cell line experiments.

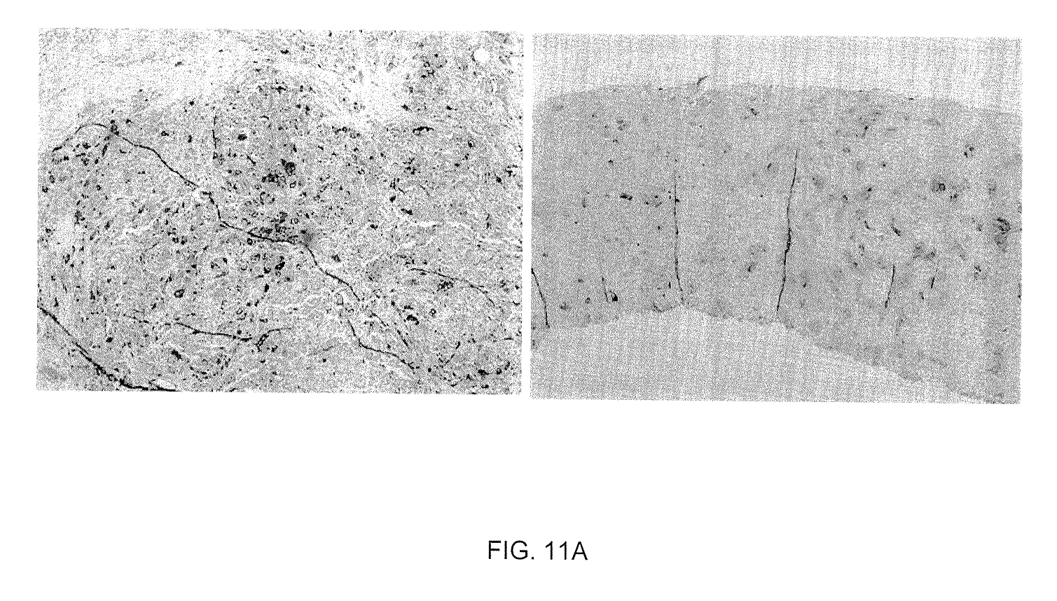

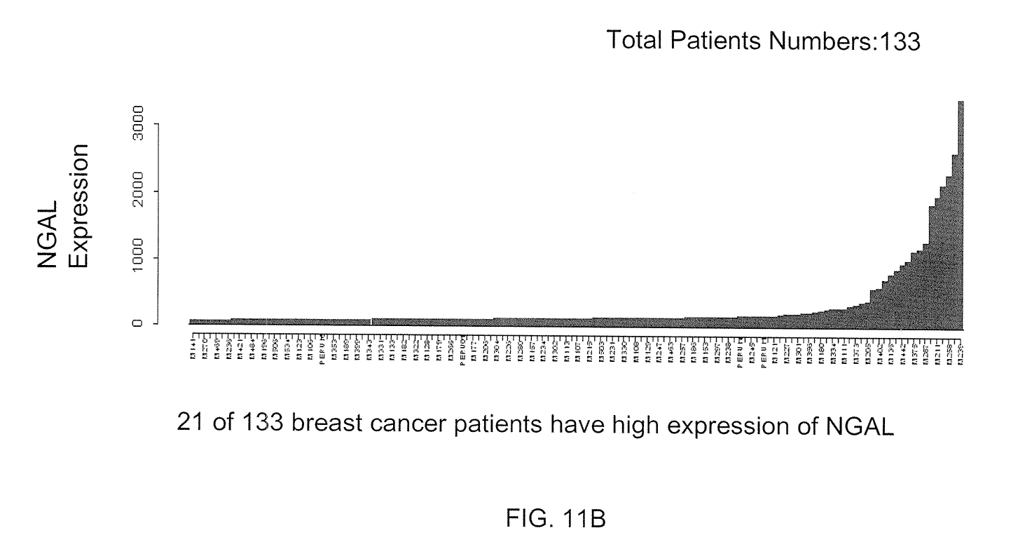

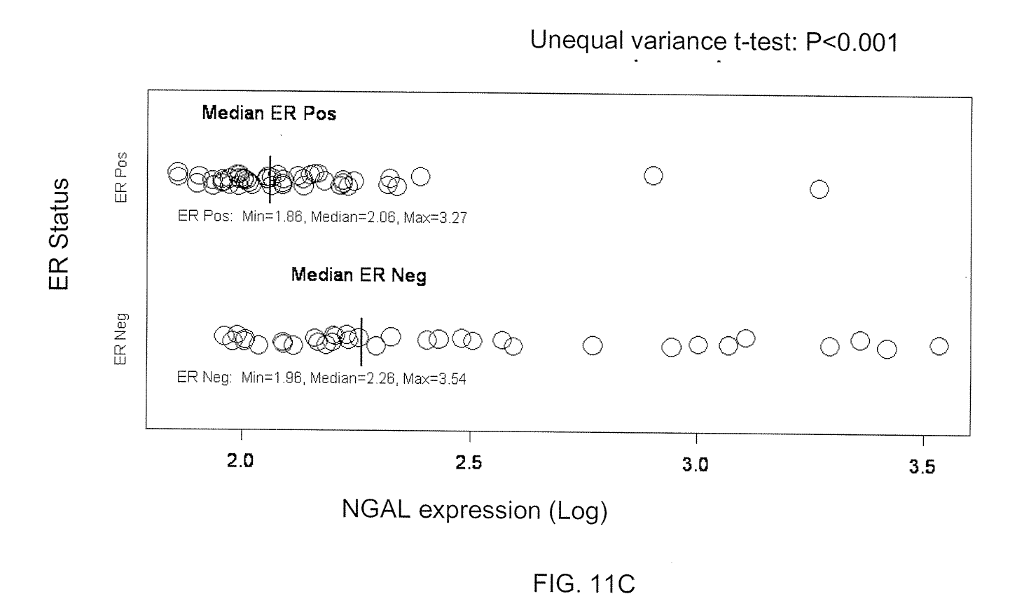

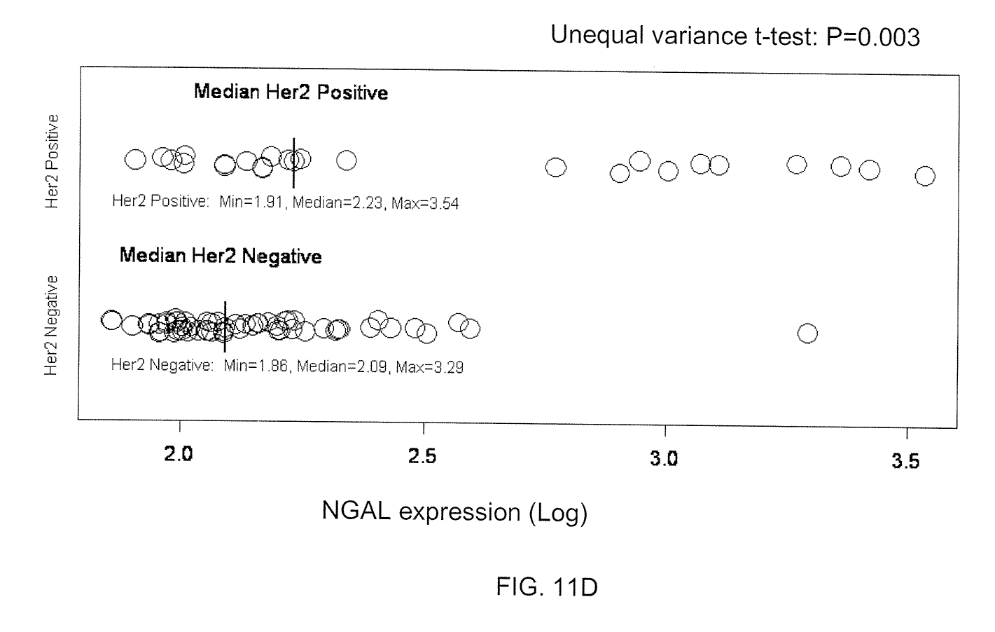

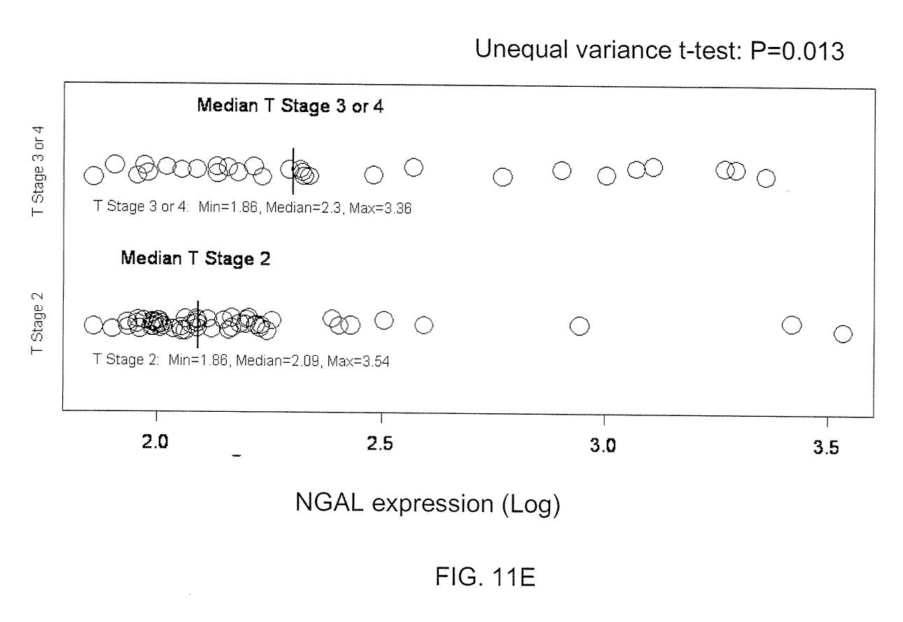

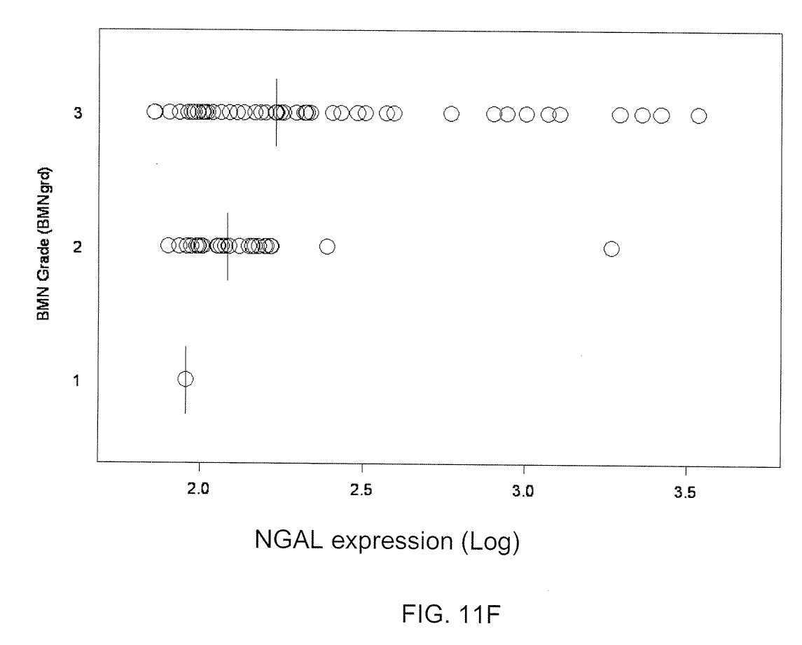

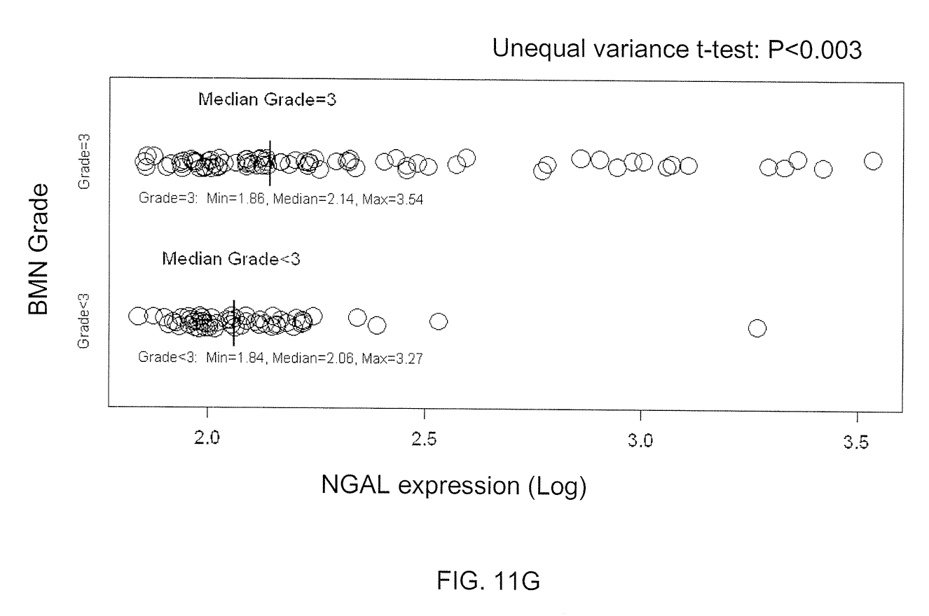

Breast tumor specimens were examined, and it was determined that breast tumors strongly expressed NGAL (FIG. 11A). A significant fraction of cells from breast patients expressed very high levels of NGAL RNA (FIG. 11B). ER(−) breast cancer cells expressed higher levels of NGAL than ER(+) cells (FIG. 11C). Importantly, high HER2/neu expression showed a strong correlation with high NGAL expression (FIG. 11D). Tumor size and grade of breast cancer aggressiveness also showed a strong correlation with high NGAL expression (FIG. 11E, 11F, and 11G).

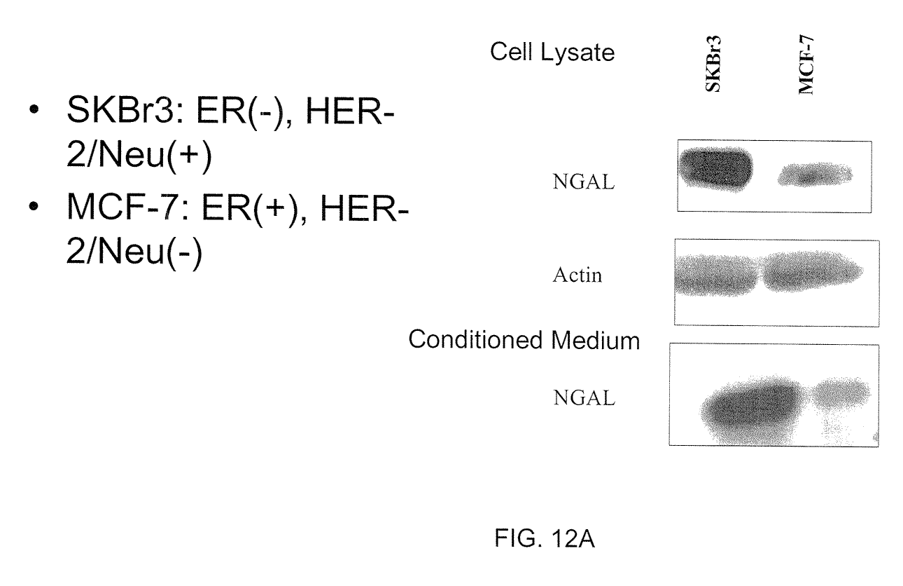

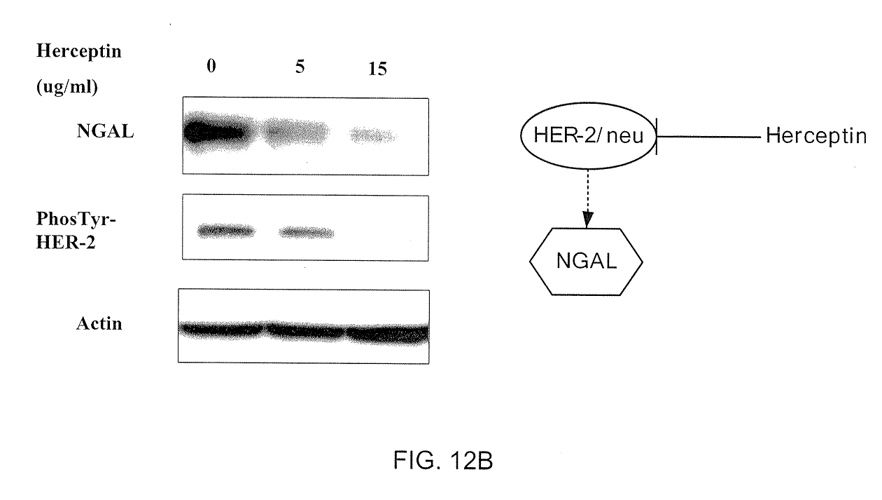

Several breast cancer cell lines were examined for NGAL expression. It was found that several cell lines including SKBr3, SKOV3, 468, MCF-7eB, 231-eB and MCF-10A expressed NGAL in their CM. The inventors focused on the HER2/neu+ SKBr3 cell line for further studies, since the MCF-7 cell line [HER-2 (−), ER(+)] had lower levels of secreted NGAL (FIG. 12A). As expected, treatment of SKBr3 cells with Herceptin, the anti-HER2 antibody, inhibited NGAL protein expression (FIG. 12B).

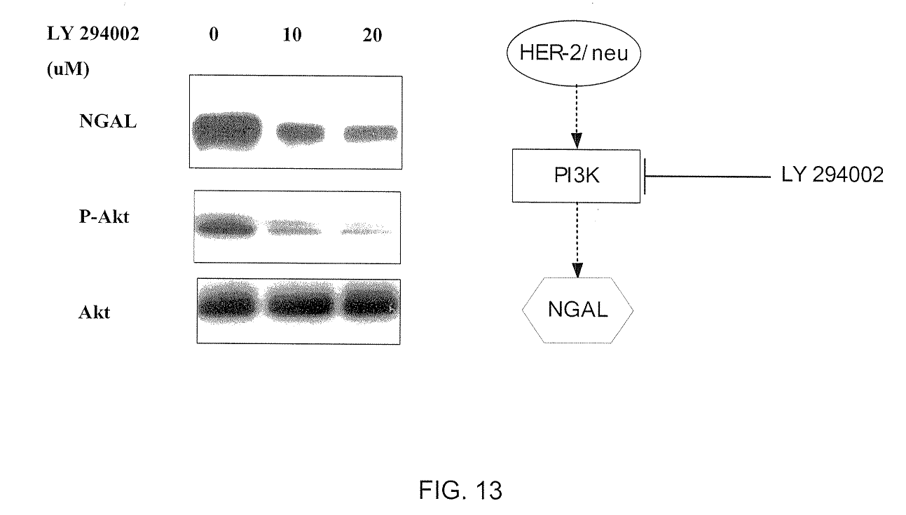

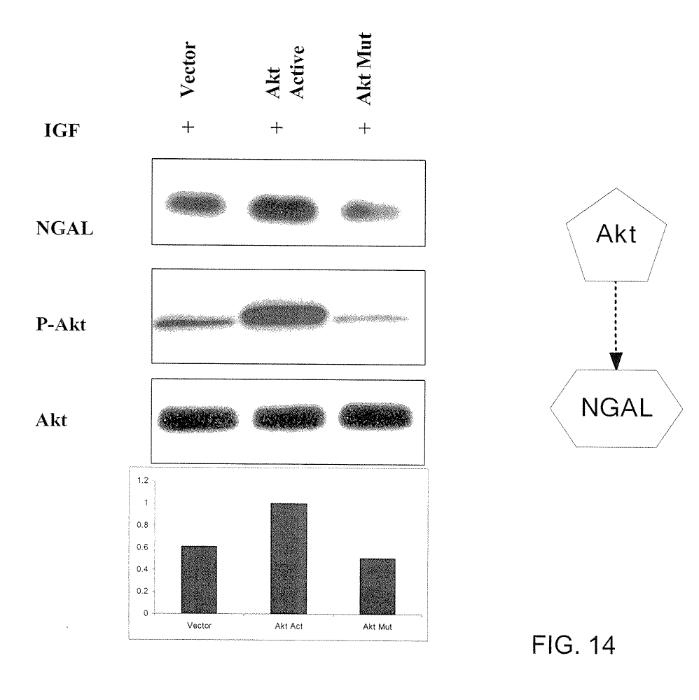

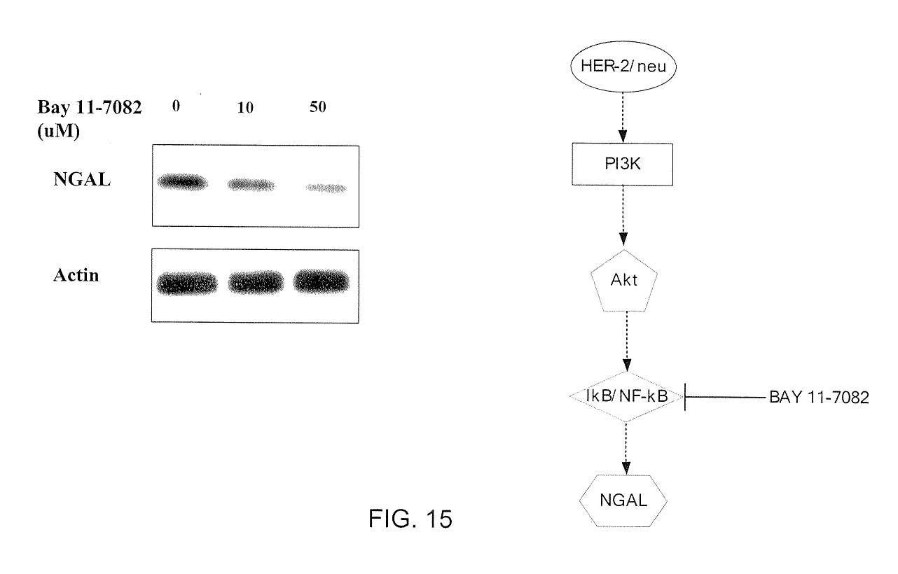

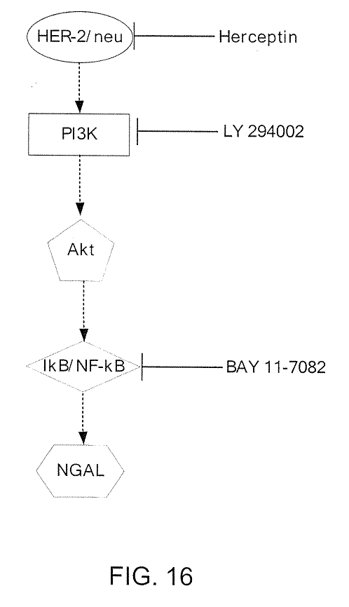

FIG. 13 shows that NGAL expression is down-regulated by the exemplary PI3K Inhibitor LY 294002. FIG. 14 demonstrates that the Akt pathway is required for NGAL expression. FIG. 15 illustrates that NFkB inhibition with the exemplary Bay 11-7082 compound attenuates NGAL expression. FIG. 15 shows NGAL expression in conditioned medium having been in the presence of particular compounds. Without meaning to be bound by the following, FIG. 16 illustrates an exemplary model of NGAL expression in breast cancer.

### Example 17

### Further Studies Associated with Cancer and Lipocalin

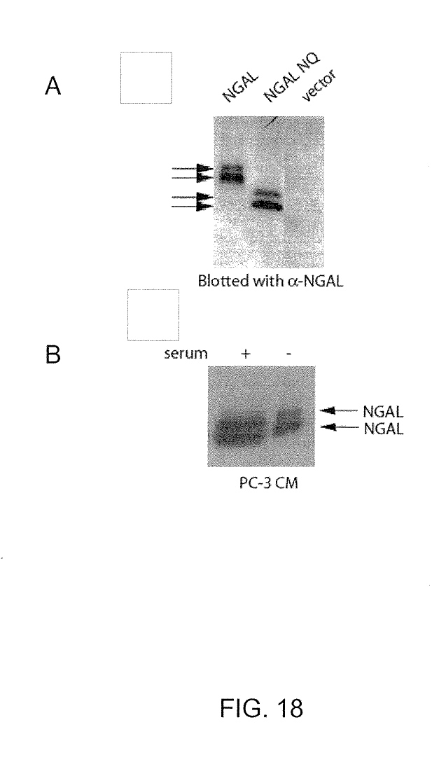

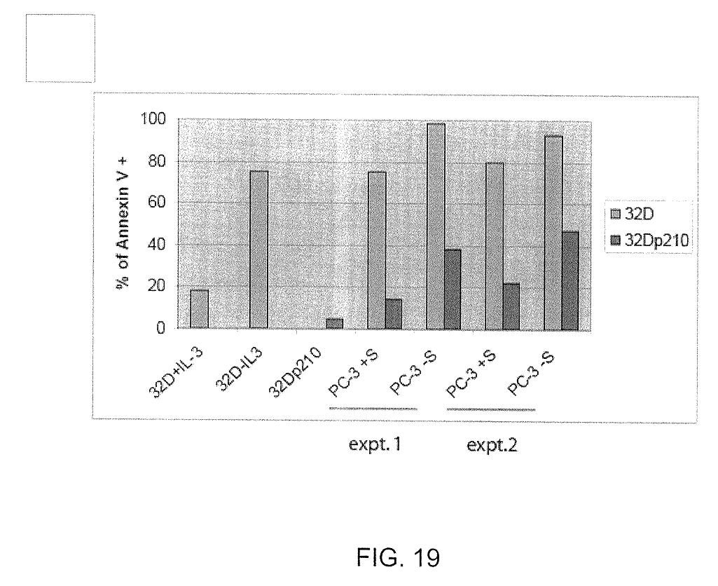

FIG. 18 shows detection of the two forms of NGAL in conditioned medium (CM) of COS-1 cells transfected with NGAL (FIG. 18A) or conditioned medium of PC-3 cells (FIG. 18B). FIG. 19 shows a high rate of apoptosis as detected by Annexin V staining, which was observed in exemplary hematopoietic 32D cells cultured with CM derived from exemplary PC-3 cells (S refers to serum). The 32D cells are considered normal, but 32Dp210 cells comprise stable expression of BCR-ABL, which effectively renders them cancerous. Experiments were performed in the presence of IL-3. In specific embodiments, high expression of BCR-ABL results in increase in expression of NGAL, which in further specific embodiments renders the cell more resistant to lipocalin. In a particular aspect of the invention, cancer cells are more resistant to the pro-apoptotic activity than a corresponding wild type cell because cancer cells have fewer receptors for lipocalin, which may be correlated to overexpression of BCR-ABL, for example.

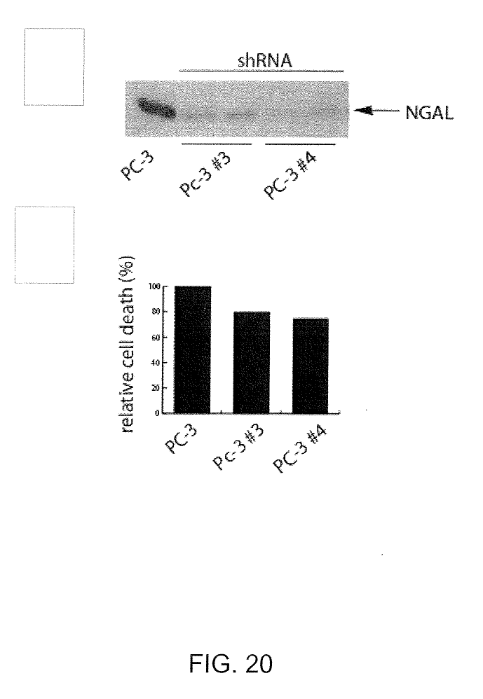

FIG. 20A shows knocking down of NGAL level using exemplary RNAi oligonucleotides (#3) and (#4) lowered the cell death inducing activity in CM of PC-3 cells. FIG. 20B illustrates % relative death in PC-3 cells transfected with either of the two exemplary RNAi oligonucleotides.

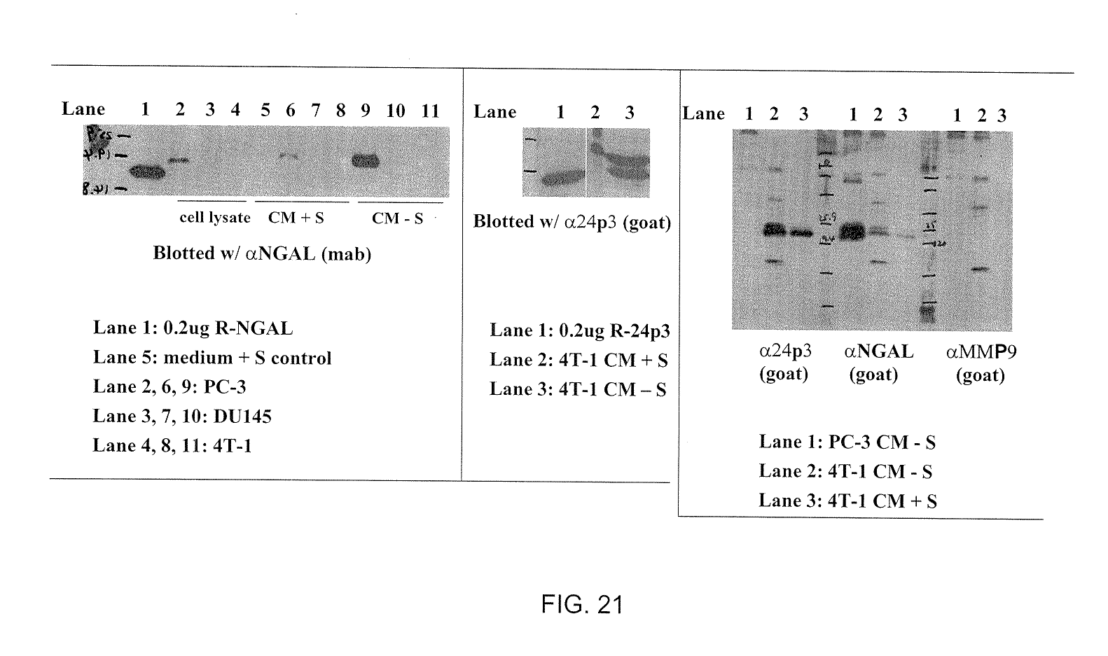

FIG. 21 illustrates expression of 24p3/NGAL in multiple tumor cell lines, including at least PC-3 cancer cells and 4T-1 breast cancer cells and which demonstrates that there are at least two forms of 24p3/NGAL therein. In the left panel, monoclonal antibody (Antibody Shop; Cat # HYB211-01) to NGAL was employed. In the middle panel, polyclonal antibodies against mouse 24p3 were employed. In the right panel, these antibodies and antibodies to matrix metalloproteinase 9 (MMP9) were employed. MMP9 is known to be stabilized in response to NGAL expression.

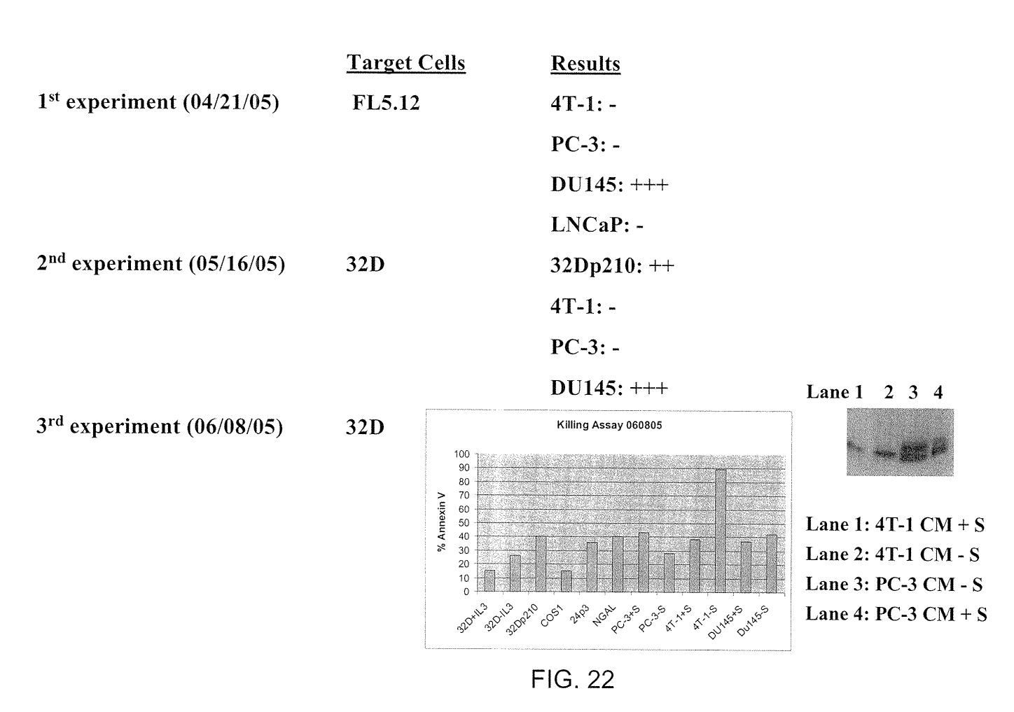

In FIG. 22, there is induction of apoptosis using conditioned media from the exemplary tumor cell lines. In the 3^{rd} experiment, for example, 32D cells (exemplary mouse hematopoietic cells) are grown in media conditioned by previous exposure to the cells noted on the abscissa of the Killing Assay figure.

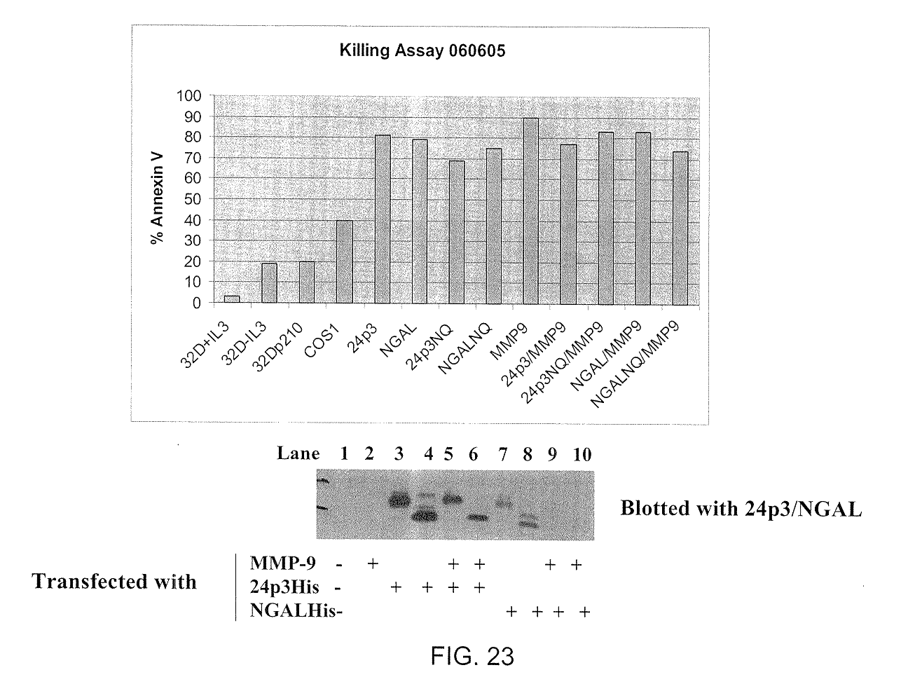

In FIG. 23, killing activity using CM from 24p3/NGAL-His transfected cells is demonstrated.

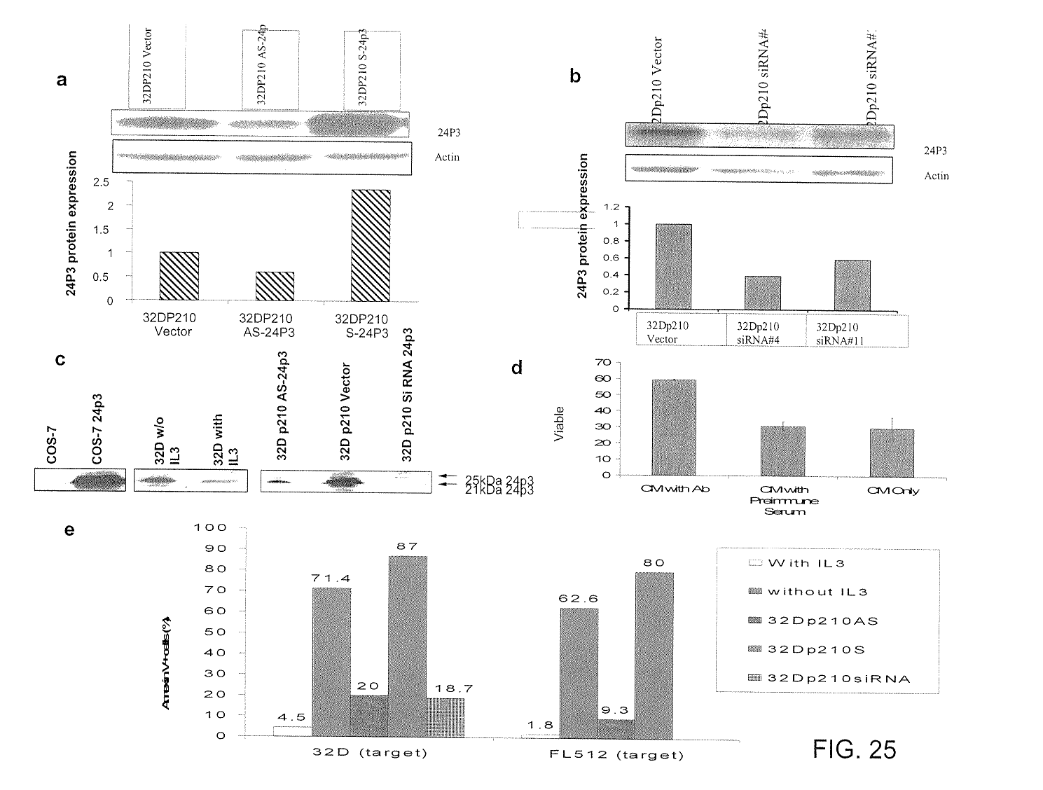

FIGS. 25A-25E demonstrate multiple experiments regarding 24p3 expression.

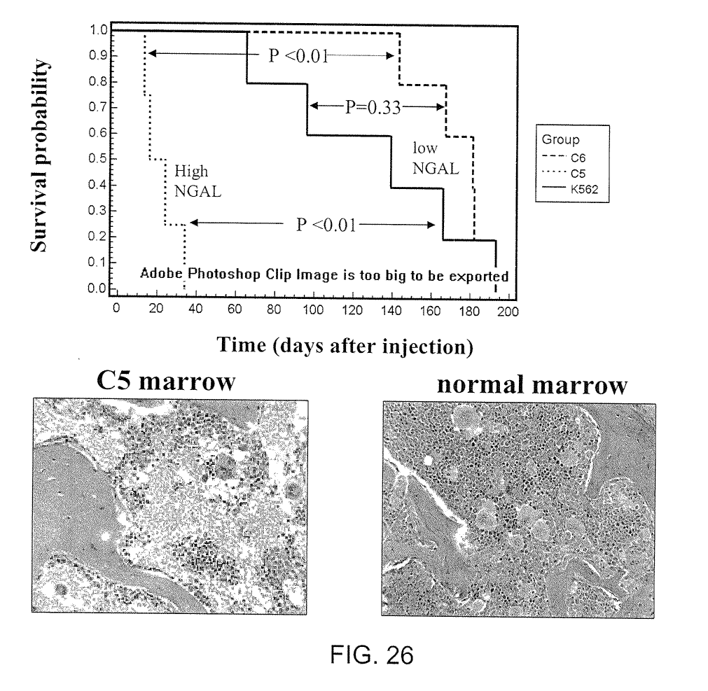

FIG. 26 shows that soft agar clones of K562 cells that express high levels of NGAL suppress hematopoiesis.

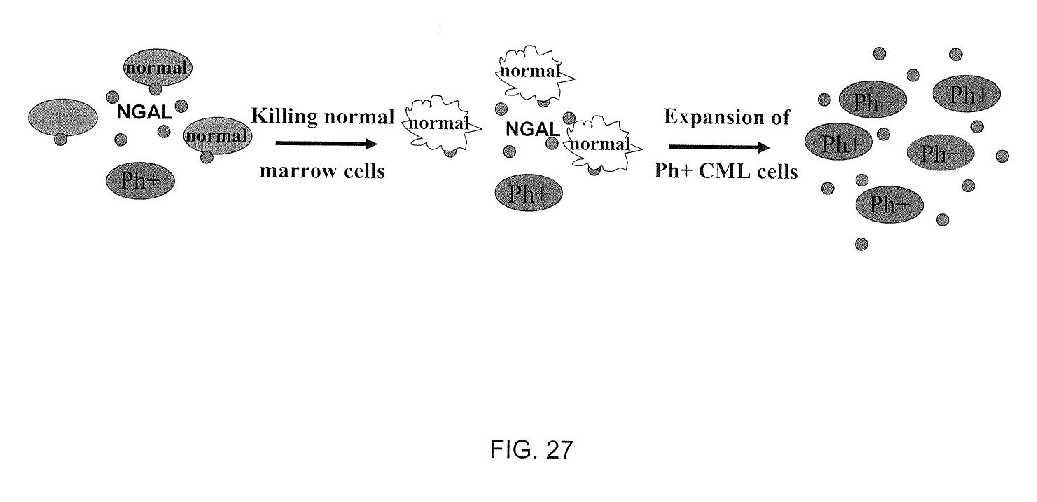

FIG. 27 illustrates an exemplary model for NGAL involvement in marrow expansion of leukemia cells. The model also applies in breast and prostate cells. Ph+ relates to Philadelphia chromosome (abnormal 22) that encodes the BCR-ABL leukemia gene seen in most CML patients.

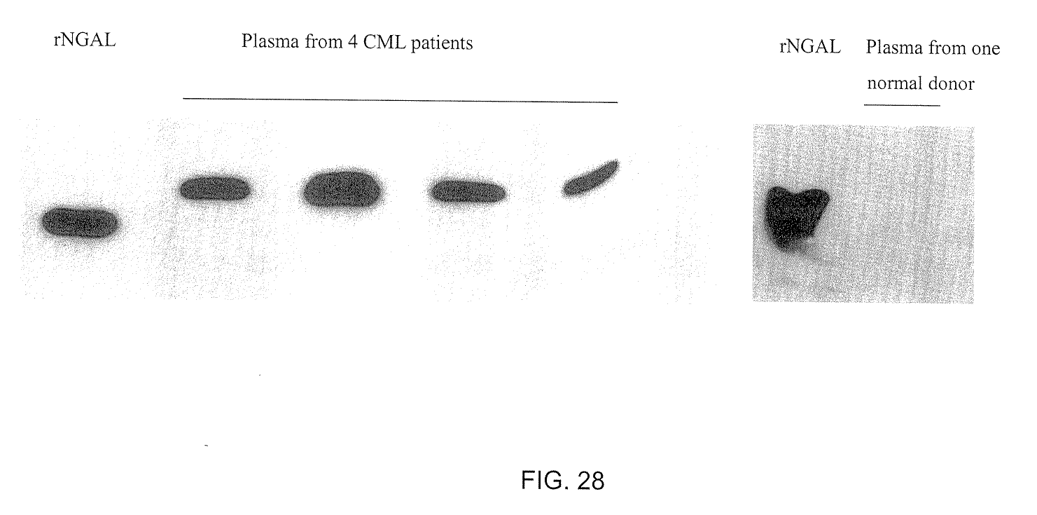

FIG. 28 provides rNGAL experiments from plasma from CML patients versus normal individuals.

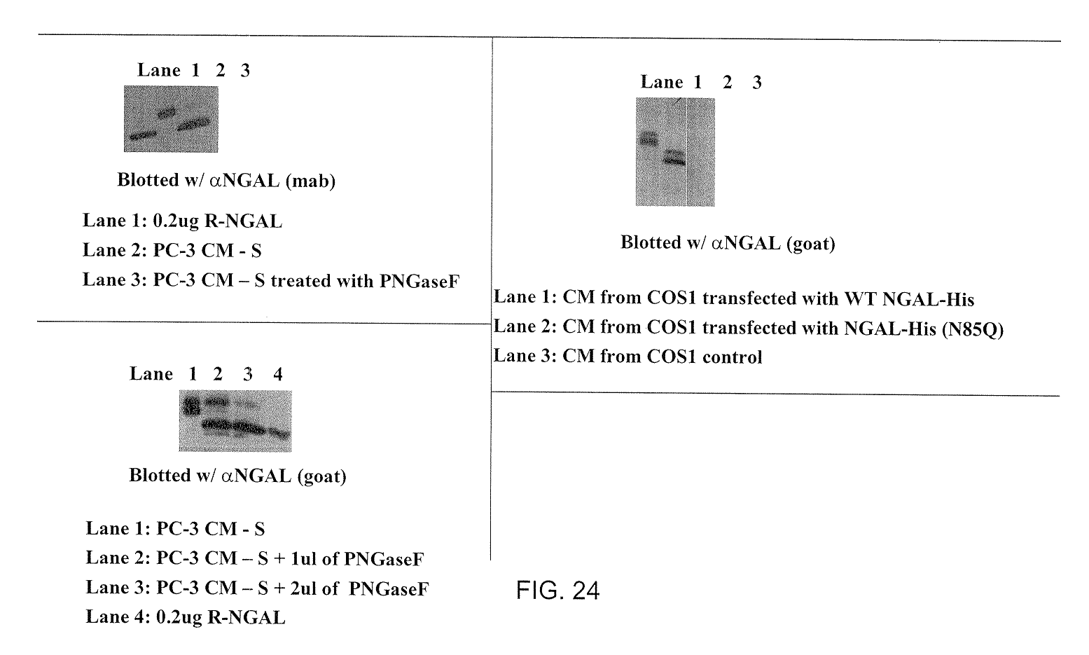

In specific embodiments, the nature of two forms of NGAL is characterized. Although the two forms of NGAL may be the result of any modification, in particular aspects it is the result of iron-binding; the result of co-factor binding; and/or the result of phosphorylation, acetylation, glycosylation, farnesylation, and/or methylation. These types of modifications may be investigated by any suitable method(s) in the art. For example, peptide mapping indicates that both forms comprise 24p3 sequence. Upon treatment of both species with protease to identify peptides, more fragments are released from the lower band (faster running 21 kD species). Mass spectrometry determined that the protein is intact, and perusal of genomic and molecular maps for the NGAL gene does not identify obvious splicing sites that would generate the noted forms, in specific embodiments of the invention. Furthermore, insect cells comprising either form of NGAL were treated with phosphatase, yet the two bands did not thereafter migrate at the same location, indicating that in specific embodiments of the invention, the difference in size between the two forms is not due to phosphorylation, in certain embodiments of the invention. Furthermore, phosphoserine antibodies do not recognize NGAL, in specific embodiments of the invention. FIG. 24 shows that both forms of 24p3/NGAL are glycosylated and that glycosylation does not effect the formation of different forms. Mutation of NGAL to render the polypeptide constitutively glycosylated still results in two forms being produced, which indicates that in specific embodiments of the invention the formation of the two forms is not directly related to glycosylation. Mass spectrometry indicated that the difference in size between the two forms is only 186 Daltons. Therefore, in specific aspects of the invention, the difference between the two forms is because of a dramatic structural change between them.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference herein.

### PUBLICATIONS

Almendro et al., J Immunol 1996 Dec. 15; 157(12):5411-21.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Banerji et al., Cell, 27:299, 1981.
Banerji et al., Cell, 35:729, 1983.
Berkhout et al., Cell, 59:273, 1989.
Blanar et al., EMBO J., 8:1139, 1989.
Bodine and Ley, EMBO J., 6:2997, 1987.
Boshart et al., Cell, 41:521, 1985.
Bosze et al., EMBO J., 5:1615, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al, J. Virology, 62:1314, 1988.
Chandler et al., Cell, 33:489, 1983.
Chang et al., Mol. Cell. Biol., 9:2153, 1989.
Chatterjee et al., Proc. Nat'l Acad. Sci. USA., 86:9114, 1989.
Choi et al., Cell, 53:519, 1988.
Cohen et al., J. Cell. Physiol., 5:75, 1987.
Costa et al., Mol. Cell. Biol., 8:81, 1988.
Cripe et al., EMBO J., 6:3745, 1987.
Culotta and Hamer, Mol. Cell. Biol., 9:1376, 1989.
Daley et al., Science 237, 532-535, 1987.
Dandolo et al., J. Virology, 47:55, 1983.
Davis et al., Biochim Biophys Acta, 1095, 145-152, 1991.
De Villiers et al., Nature, 312:242, 1984.
Deschamps et al., Science, 230:1174, 1985.
Devireddy et al., Science 293, 829-834, 2001.
Druker, Oncogene 21, 8541-8546, 2002.
Eaves et al., Leuk. Res. 22, 1085-1096, 1998.
Edbrooke et al., Mol. Cell. Biol., 9:1908, 1989.
Edlund et al., Science, 230:912, 1985.
EL-Ouiiaghli et al, Blood 102, 3786-3792, 2003.
Feng and Holland, Nature, 334:6178, 1988.
Fernandez et al., Clin Cancer Res. 11(15):5390-5, 2005.
Firak and Subramanian, Mol. Cell. Biol., 6:3667, 1986.
Flower et al., Biochem Biophys Res Commun. 180, 69-74, 1991.
Foecking and Hofstetter, Gene, 45:101, 1986.
Fujita et al., Cell, 49:357, 1987.
Gilles et al., Cell, 33:717, 1983.
Gloss et al., EMBO J., 6:3735, 1987.
Godbout et al., Mol. Cell. Biol., 8:1169, 1988.
Goetz et al., Mol Cell. 10, 1033-1043, 2002.
Goodbourn and Maniatis, Proc. Nat'l Acad. Sci. USA, 85:1447, 1988.
Goodbourn et al., Cell, 45:601, 1986.
Greene et al., Immunology Today, 10:272, 1989.
Grosschedl and Baltimore, Cell, 41:885, 1985.
Guo et al., Leukem. 16, 2447-2453, 2002.
Haslinger and Karin, Proc. Nat'l Acad. Sci. USA., 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Huang et al., Cell, 27:245, 1981.
Hug et al., Mol Cell Biol, August; 8(8):3065-79, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Karin et al., Mol. Cell. Biol., 7:606, 1987.
Katinka et al., Cell, 20:393, 1980.
Katinka et al., Nature, 290:720, 1981.
Kawamoto et al., Mol. Cell. Biol., 8:267, 1988.
Kiledjian et al., Mol. Cell. Biol., 8:145, 1988.
Kjeldsen et al., Biochim Biophys Acta 1482, 272-283, 2000.
Kjeldsen et al., J Biol Chem. 268, 10425-10432, 1993.
Klamut et al., Mol. Cell. Biol., 10:193, 1990.
Koch et al., Mol. Cell. Biol., 9:303, 1989.
Kraus et al., FEBS Lett., May 29; 428(3): 165-70, 1998.
Kriegler and Botchan, In: Eukaryotic Viral Vectors, Y. Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kriegler et al., In: Gene Expression, D. Hamer and M. Rosenberg, eds., New York: Alan R. Liss, 1983.
Kuhl et al., Cell, 50:1057, 1987.
Kunz et al., Nucl. Acids Res., 17:1121, 1989.
Lareyre et al., J Biol Chem, March 19; 274(12):8282-90, 1999.
Larsen et al., Proc. Nat'l Acad. Sci. USA., 83:8283, 1986.
Laspia et al., Cell, 59:283, 1989.
Latimer et al., Mol. Cell. Biol., 10:760, 1990.
Lee et al., Mol. Endocrinol., 2: 404-411, 1988.
Lee et al., Nature, 294:228, 1981.
Lee et al., DNA Cell Biol., November; 16(11):1267-75, 1997.
Levinson et al., Nature, 295:79, 1982.
Lin et al., Mol. Cell. Biol., 10:850, 1990.
Lin et al., Oncogene 20, 1873-1881, 2001.
Ling et al., Cancer Res. 63, 298-303, 2003.
Liu et al., Mol Reprod Dev. 46, 507-514, 1997.
Luria et al., EMBO J., 6:3307, 1987.
Lusky and Botchan, Proc. Nat'l Acad. Sci. USA., 83:3609, 1986.
Lusky et al., Mol. Cell. Biol., 3:1108, 1983.
Majors and Vammus, Proc. Nat'l Acad. Sci. USA., 80:5866, 1983.
Matulonis et al., Blood 85, 2507-2515, 1995.
McCubrey et al., Oncogene 8, 2905-2915, 1993.
McNeall et al., Gene, 76:81, 1989.
Miksicek et al., Cell, 46:203, 1986.
Mordacq and Linzer, Genes and Dev., 3:760, 1989.
Moreau et al., Nul. Acids Res., 9:6047, 1981.
Muesing et al., Cell, 48:691, 1987.
Ng et al., Nuc. Acids Res., 17:601, 1989.
Nomoto et al., Gene, August 20; 236(2):259-71, 1999.
Olofsson et al., Blood 55, 983-991, 1980b.
Olofsson et al., Blood 55, 975-982, 1980a.
Ondek et al., EMBO J., 6:1017, 1987.
Ornitz et al., Mol. Cell. Biol., 7:3466, 1987.
Palmiter et al., Nature, 300:611, 1982.
Pech et al., Mol. Cell. Biol., 9:396, 1989.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Nat'l Acad. Sci. USA., 82:1020, 1985.
Porton et al., Mol. Cell. Biol., 10:1076, 1990.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Resendez Jr. et al., Mol. Cell. Biol., 8:4579, 1988.
Ripe et al., Mol. Cell. Biol., 9:2224, 1989.
Rittling et al., Nucl. Acids Res., 17:1619, 1989.
Rosen et al., Cell, 41:813, 1988.
Salomoni et al., Blood 96, 676-684, 2000.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Skold et al., Blood 93, 849-856, 1999.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stoesz S. P. et al., Int. J. Cancer. 79 (6):565-72 1998.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Treisman, Cell, 42:889, 1985.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsumaki et al., J Biol Chem September 4; 273(36):22861-4, 1998.
Tyndall et al., Nuc. Acids. Res., 9:6231, 1981.
Vannice and Levinson, J. Virology, 62:1305, 1988.
Vasseur et al., Proc. Nat'l Acad. Sci. USA., 77:1068, 1980.
Wang and Calame, Cell, 47:241, 1986.
Weber et al., Cell, 36:983, 1984.
Weinberger et al. Mol. Cell. Biol., 8:988, 1984.
Winoto and Baltimore, Cell, 59:649, 1989.
Wiznerowicz et al., J. Virol. 77, 8957-8961, 2003.
Wu et al., Biochem Biophys Res Commun., April 7; 233(1):221-6, 1997.
Xie et al., Oncogene 21, 7137-46, 2002.
Yousefi et al., Cell Death and Differentiation 9, 595-597, 2002.
Yutzey et al., Mol. Cell. Biol., 9:1397, 1989.
Yutzey et al. Mol. Cell. Biol., 9:1397, 1989.
Zhao-Emonet et al., Biochim Biophys Acta, November 8; 1442(2-3):109-19, 1998.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of inhibiting secretion of lipocalin from a cell and/or targeting a secreted lipocalin from a cell of an individual, comprising the step of administering to the individual a therapeutically effective amount of a lipocalin-inhibiting substance.

2. The method of claim 1, wherein the lipocalin-inhibiting substance comprises a small molecule, an antibody, a DNA, an RNA, a polypeptide, a peptide, a combination thereof, or a mixture thereof.

3. The method of claim 1, wherein the lipocalin-inhibiting substance comprises an antibody.

4. The method of claim 3, wherein the antibody is a monoclonal antibody.

5. The method of claim 3, wherein the antibody is a humanized antibody.

6. The method of claim 1, wherein the lipocalin-inhibiting substance comprises antisense RNA, siRNA, or both.

7. The method of claim 1, wherein the lipocalin-inhibiting substance is identified by the method of claim 22.

8. The method of claim 1, wherein the individual has cancer.

9. The method of claim 8, wherein the cancer is leukemia, breast cancer, or prostate cancer.

10. The method of claim 8, wherein the cancer is leukemia.

11. The method of claim 10, wherein the leukemia is chronic myeloid leukemia.

12. The method of claim 8, wherein the cancer is breast cancer.

13. The method of claim 8, wherein the cancer is prostate cancer.

14. The method of claim 1, wherein the lipocalin is a modified form of lipocalin.

15. The method of claim 14, wherein the modified form of lipocalin is about 21 kDa.

16. The method of claim 1, wherein the administering step comprises injection.

17. The method of claim 1, wherein the lipocalin-inhibiting substance comprises a vector.

18.-21. (canceled)

22. A method of screening for an agent that inhibits secretion of lipocalin from a cell or that targets secreted lipocalin from a cell, comprising the steps of:
obtaining lipocalin;
providing a test compound suspected of binding lipocalin; and
assaying for the modulation of lipocalin by said test compound, wherein when said test compound modulates lipocalin, said test compound is said agent.

23. The method of claim 22, further comprising the manufacturing of the agent.

24. The method of claim 22, wherein the modulation is further defined as the binding of lipocalin by the test compound.

25. The method of claim 22, wherein the modulation of lipocalin by the test compound renders lipocalin biologically inactive.

26. The method of claim 22, further defined as:
providing a cell that secretes lipocalin into a medium;
providing a test compound to the cell, the medium or both; and
assaying the medium for the presence of lipocalin, assaying modulation of lipocalin by the test compound, or both.

27. The method of claim 22, wherein the test compound is an antibody, a small molecule, a nucleic acid, a polypeptide, a peptide, or a mixture thereof.

28.-37. (canceled)

38. The method of claim 32, wherein the individual is treated with an additional cancer therapy.

39. The method of claim 38, wherein the additional cancer therapy comprises chemotherapy, transplant, radiation, surgery, gene therapy, hormone therapy, immunotherapy, or a combination thereof.

40.-43. (canceled)

44. A method of protecting a non-cancerous cell from destruction by lipocalin in an individual, comprising the step of delivering to the individual an agent identified by the method of claim 22.

45. The method of claim 44, wherein the non-cancerous cell is a bone marrow cell.

46. A method of preventing proliferation of one or more cancer cells in an individual, comprising the step of delivering to the individual an agent that inhibits secretion of lipocalin or that targets secreted lipocalin from one or more cancer cells.

47. The method of claim 46, further defined as preventing destruction of a non-cancerous cell by secreted lipocalin.

48.-55. (canceled)
